(19) United States
(12) Patent Application Publication
(10) Pub. No.: US 2010/0311756 A1
(43) Pub. Date: Dec. 9, 2010

(54) METHODS FOR DELAYING THE PROGRESSION OF AT LEAST ONE OF CARDIAC HYPERTROPHY, CARDIAC REMODELING OR LEFT VENTRICULAR FUNCTION OR THE ONSET OF HEART FAILURE IN SUBJECTS IN NEED OF TREATMENT THEREOF

(75) Inventors: Lin Zhao, Vernon Hills, IL (US); Robert Hamlin, Columbus, OH (US); Yingjie Chen, Roseville, MN (US)

Correspondence Address: POLSINELLI SHUGHART PC, 700 W. 47TH STREET, SUITE 1000, KANSAS CITY, MO, 64112-1802, US

(73) Assignee: TAKEDA PHARMACEUTICALS NORTH AMERICA, INC., Deerfield, IL (US)

(21) Appl. No.: 12/689,266

(22) Filed: Jan. 19, 2010

Related U.S. Application Data
(60) Provisional application No. 61/146,518, filed on Jan. 22, 2009.

(51) Int. Cl. A61K 31/53; A61K 31/426; A61K 31/415; A61K 31/4439; A61K 31/10; A61P 9/12; A61P 9/10; A61P 9/00

(52) U.S. Cl. 514/246; 514/365; 514/406; 514/340; 514/709

(57) ABSTRACT

The present invention relates to methods for reducing the progression of at least one of cardiac hypertrophy, cardiac remodeling or left ventricular dysfunction following a cardiac insult in a subject in need of treatment thereof. Moreover, the present invention also relates to methods of delaying the onset of heart failure symptoms or reducing the incidence of cardiovascular events following a cardiac insult in a subject in need of treatment thereof.

### RELATED APPLICATION INFORMATION

This application claims priority to U.S. Ser. No. 61/146,518 filed on Jan. 22, 2009, the contents of which are herein incorporated by reference.

### FIELD OF THE INVENTION

In one aspect, the present invention relates to methods for reducing the progression of at least one of cardiac hypertrophy, cardiac remodeling or left ventricular dysfunction following a cardiac insult in a subject in need of treatment thereof. In another aspect, the present invention relates to methods of delaying the onset of heart failure symptoms or reducing the incidence of cardiovascular events following a cardiac insult in a subject in need of treatment thereof.

### BACKGROUND OF THE INVENTION

Heart failure (HF) is a major and growing public health problem in the United States. Approximately 5 million patients in this country have HF, and over 550 000 patients are diagnosed with HF for the first time each year (See, American Heart Association, Heart Disease and Stroke Statistics: 2005 Update. Dallas, Tex.: American Heart Association; 2005). Heart failure is the primary reason for 12 to 15 million office visits and 6.5 million hospital days each year (O'Connell J B, et al., J. Heart Lung Transplant, 13;S107-12 (1994)).

Heart failure is a complex clinical syndrome that can result from any structural or functional cardiac disorder that impairs the ability of the ventricle to fill with or eject blood (See, Hunt, S., “ACC/AHA 2005 Guideline Update for the Diagnosis and Management of Chronic Heart Failure in the adult: A Report of the American College of Cardiology/American Heart Association Task Force on Practice Guidelines (Writing Committee to Update the 2001 Guidelines for the Evaluation and Management of Heart Failure),” J. Am. Coll. Cardiol., 46:e1-e82 (2005)). The cardinal manifestations of HF are dyspnea and fatigue (which may limit exercise tolerance) and fluid retention (which may lead to pulmonary congestion and peripheral edema). Id. Both abnormalities can impair the functional capacity and quality of life of affected subjects.

Heart failure may result from disorders of the pericardium, myocardium, endocardium or great vessels, but the majority of patients with HF have symptoms due to an impairment of left ventricular (LV) myocardial function, Id. The causes of HF include coronary artery disease, hypertension and dilated cardiomyopathy. Id. As many as 30% of patients with dilated cardiomyopathy may have a genetic cause (See, Francis, G S, et al., “Pathophysiology of congestive heart failure secondary to congestive and ischemic cardiomyopathy. In: Sahver J A, ed. Cardiomyopathies: Clinical Presentation, Differential Diagnosis and Management. Philadelphia, Pa.: F A Davis; 1988:57-74). Another common cause of HF is valvular heart disease (See, Hunt, S., “ACC/AHA 2005 Guideline Update for the Diagnosis and Management of Chronic Heart Failure in the adult: A Report of the American College of Cardiology/American Heart Association Task Force on Practice Guidelines (Wilting Committee to Update the 2001 Guidelines for the Evaluation and Management of Heart Failure),” J. Am. Coll. Cardiol., 46:e1-e82 (2005)). In fact, nearly any form of heart disease may ultimately lead to HF Id.

HF is not the same as cardiomyopathy or to LV dysfunction, Cardiomyopathy and LV dysfunction provide structural or functional reasons for the development of HF. Id. In contrast, HF refers to a clinical syndrome that is characterized by specific symptoms (such as, dyspnea and fatigue) in the medical history and signs (such as edema, rales) on the physical examination. Id.

LV dysfunction begins with some injury to, or stress on, the myocardium and is generally a progressive process, even in the absence of a new identifiable insult to the heart. Id. The principal manifestation of such progression is a change in the geometry and structure of the LV, such that the chamber dilates and/or hypertrophies and becomes more spherical—a process referred to as cardiac remodeling, Id. This change in chamber size and structure not only increases the hemodynamic stresses on the walls of the failing heart and depresses its mechanical performance but may also increase regurgitant flow through the mitral valve. Id. These effects, in turn, serve to sustain and exacerbate the remodeling process. Id. Cardiac remodeling generally precedes the development of symptoms (sometimes by months or even years), continues after the appearance of symptoms, and contributes substantially to worsening of symptoms despite treatment. Progression of coronary artery disease, diabetes mellitus, hypertension or the onset of atrial fibrillation may also contribute to the progression of HF. Id. The development of structural abnormalities in the heart can lead to 1 of 3 possible outcomes: 1) patients die before developing symptoms (if they are in Stage A or B (See, below); 2) patients develop symptoms controlled by treatment, or 3) patients die of progressive HF. Id. Sudden death can interrupt this course at any time. Id.

The development of HF can be characterized by considering 4 stages of the disease. The first stage, Stage A, is a subject at high risk for HF but without structural heart disease or symptoms of HF (for example, these are patients with hypertension, atherosclerotic disease, diabetes, obesity, metabolic syndrome or patients using cardiotoxins). The second stage, Stage B, is a subject having structural heart disease but without signs or symptoms of HF (for example, these are patients who have previously had a myocardial infarction, exhibit LV remodeling including LV hypertrophy and low ejection fraction (EF), and patients with asymptomatic valvular disease). The third stage, Stage C, is a subject having structural heart disease with prior or current symptoms of HF (for example, these are patients who have known structural heart disease and exhibit shortness of breath and fatigue and have reduced exercise tolerance). The fourth and final stage, Stage D, is refractory HF requiring specialized interventions (for example, patients who have marked symptoms at rest despite maximal medical therapy (namely, those who are recurrently hospitalized or cannot be safely discharged from the hospital without specialized interventions). Each of these Stages A-D is and the therapy for each of these stages is described in Hunt, S., “ACC/AHA 2005 Guideline Update for the Diagnosis and Management of Chronic Heart Failure in the Adult: A Report of the American College of Cardiology/American Heart Association Task Force on Practice Guidelines (Writing Committee to Update the 2001 Guidelines for the Evaluation and Management of Heart Failure),” J. Am, Coll. Cardiol, 46:e1-e82 (2005).

A number of drug therapies are known for preventing or delaying the onset of HF at its various stages (namely, Stages A-D). These include vasodilators, angiotensin II receptor antagonists, angiotensin converting enzyme inhibitors, aldosterone antagonists, diuretics, hydralazine/nitrates, antithrombolytic agents, β-adrenergic receptor antagonists, α-adrenergic receptor antagonists, calcium channel blockers, etc. Of course, any drug used for treatment may result in side effects. In addition, the currently available therapies are insufficient in preventing HF as HF remains a growing public health problem.

There is a need in the art for new mono therapies or combination therapies (such as with at least one of the drug therapies listed above) which exhibit reduced side effects that can be used to reduce the progression of at least one of cardiac hypertrophy, cardiac remodeling or left ventricular dysfunction in a subject following a cardiac insult. Moreover, there is also a need in the art for new mono therapies or combination therapies (such as with at least one of the drug therapies listed above) that can be used to delay the onset of heart failure symptoms or reduce the incidence of cardiovascular events following a cardiac insult in a subject.

### SUMMARY OF THE PRESENT INVENTION

In one embodiment, the present invention relates to a method for reducing the progression of at least one of cardiac hypertrophy, cardiac remodeling or left ventricular dysfunction following a cardiac insult in a subject in need of treatment thereof. The method comprises the step of:

administering to the subject in need of treatment thereof a therapeutically effective amount of at least one compound to reduce the progression of at least one of cardiac hypertrophy, cardiac remodeling or left ventricular dysfunction following a cardiac insult, wherein said at least one compound is a xanthine oxidoreductase inhibitor or a pharmaceutically acceptable salt thereof.

In the above method, the xanthine oxidoreductase inhibitor can be selected from the group consisting of: 2-[3-cyano-4-(2-methylpropoxy)phenyl]-4-methylthiazole-5-carboxylic acid, 2-[3-cyano-4-(3-hydroxy-2-methylpropoxy)phenyl]-4-methyl-5-thiazolecarboxylic acid, 2-[3-cyano-4-(2-hydroxy-2-methylpropoxy)phenyl]-4-methyl-5-thiazolecarboxylic acid, 2-(3-cyano-4-hydroxyphenyl)-4-methyl-5-thiazolecarboxylic acid, 2-[4-(2-carboxypropoxy)-3-cyanophenyl]-4-methyl-5-thiazolecarboxylic acid, 1-(3-cyano-4-(2,2-dimethylpropoxy)phenyl)-1H-pyrazole-4-carboxylic acid, 1-3-cyano-4-(2,2-dimethylpropoxy)phenyl]-1H-pyrazole-4-carboxylic acid, pyrazolo [1,5-a]-1,3,5-triazin-4-(1H)-one, 8-[3-methoxy-4-(phenylsulfinyl)phenyl]-sodium salt (±), 3-(2-methyl-4-pyridyl)-5-cyano-4-isobutoxyphenyl)-1,2,4-triazole and a pharmaceutically acceptable salt thereof.

In the above method, the cardiac insult can be myocardial infarction, chronic hypertension or combinations thereof.

Additionally, in the above method, the compound can be administered to the subject within about 1 minute to about 16 days after the myocardial infarction.

In yet another embodiment, the present invention relates to a method for reducing the progression of at least one of cardiac hypertrophy, cardiac remodeling or left ventricular dysfunction following a cardiac insult in a subject in need of treatment thereof. The method comprises the step of:

administering to the subject in need of treatment thereof a therapeutically effective amount of at least one compound to delay the progression of at least one of cardiac hypertrophy, cardiac remodeling or left ventricular dysfunction following a cardiac insult, wherein said at least one compound is comprises the formula:

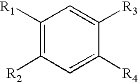

wherein R₁ and R₂ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, a phenylsulfinyl group or a cyano (—CN) group;

wherein R₃ and R₄ are each independently a hydrogen or A, B, C or D as shown below:

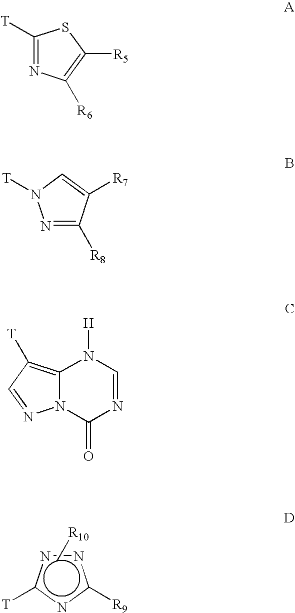

wherein T connects A, B, C or D to the aromatic ring shown above at R₁, R₂, R₃ or R₄,

wherein R₅ and R₆ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;

wherein R₇ and R₈ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;

wherein R₉ is an unsubstituted pyridyl group or a substituted pyridyl group; and

wherein R₁₀ is a hydrogen or a lower alkyl group, a lower alkyl group substituted with a pivaloyloxy group and in each case, R₁₀ bonds to one of the nitrogen atoms in the 1,2,4-triazole ring shown above.

In one aspect, the compound in the above method can be 2-[3-cyano-4-(2-methylpropoxy)phenyl]-4-methylthiazole-5-carboxylic acid or a pharmaceutically acceptable salt thereof. In another aspect, the compound in the above method can be 2-[3-cyano-4-(3-hydroxy-2-methylpropoxy)phenyl]-4-methyl-5-thiazolecarboxylic acid or a pharmaceutically acceptable salt thereof. In still yet another aspect, the compound in the above method can be 2-[3-cyano-4-(2-hydroxy-2-methylpropoxy)phenyl]-4-methyl-5-thiazolecarboxylic acid or a pharmaceutically acceptable salt thereof. In still yet another aspect, the compound in the above method can be 2-(3-cyano-4-hydroxyphenyl)-4-methyl-5-thiazolecarboxylic acid or a pharmaceutically acceptable salt thereof. In still yet another aspect, the compound in the above method can be 2-[4-(2-carboxypropoxy)-3-cyanophenyl]-4-methyl-5-thiazolecarboxylic acid or a pharmaceutically acceptable salt thereof. In still yet another aspect, the compound in the above method can be 1-3-cyano-4-(2,2-dimethylpropoxy)phenyl]-1H-pyrazole-4-carboxylic acid or a pharmaceutically acceptable salt thereof. In still yet another aspect, the compound in the above method can be pyrazolo [1,5-a]-1,3,5-triazin-4-(1H)-one, 8-[3-methoxy-4-(phenylsulfinyl)phenyl]-sodium salt (±). In still yet another aspect, the compound in the above method can be 3-(2-methyl-4-pyridyl)-5-cyano-4-isobutoxyphenyl)-1,2,4-triazole or a pharmaceutically acceptable salt thereof.

In the above method, the cardiac insult can be myocardial infarction, chronic hypertension or combinations thereof.

Additionally, in the above method, the compound can be administered to the subject within about 1 minute to about 16 days after the myocardial infarction.

In yet another embodiment, the present invention relates to a method for reducing the progression of at least one of cardiac hypertrophy, cardiac remodeling or left ventricular dysfunction following a cardiac insult in a subject in need of treatment thereof. The method comprises the step of:

administering to the subject in need of treatment thereof a therapeutically effective amount of at least one compound to delay the progression of at least one of cardiac hypertrophy, cardiac remodeling or left ventricular dysfunction following a cardiac insult, wherein said at least one compound is comprises the formula:

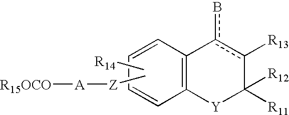

wherein R₁₁ and R₁₂ are each independently a hydrogen, a substituted or unsubstituted lower alkyl group, a substituted or unsubstituted phenyl, or R₁₁ and R₁₂ may together form a four- to eight-membered carbon ring together with the carbon atom to which they are attached;

wherein R₁₃ is a hydrogen or a substituted or unsubstituted lower alkyl group;

wherein R₁₄ is one or two radicals selected from a group consisting of a hydrogen, a halogen, a nitro group, a substituted or unsubstituted lower alkyl, a substituted or unsubstituted phenyl, —OR₁₆ and —SO₂NR₁₇R_{17′}, wherein R₁₆ is a hydrogen, a substituted or unsubstituted lower alkyl, a phenyl-substituted lower alkyl, a carboxymethyl or ester thereof, a hydroxyethyl or ether thereof, or an allyl; R₁₇ and R_{17′} are each independently a hydrogen or a substituted or unsubstituted lower alkyl;

wherein R₁₅ is a hydrogen or a pharmaceutically active ester-forming group;

wherein A is a straight or branched hydrocarbon radical having one to five carbon atoms;

wherein B is a halogen, an oxygen, or an ethylenedithio;

wherein Y is an oxygen, a sulfur, a nitrogen or a substituted nitrogen;

wherein Z is an oxygen, a nitrogen or a substituted nitrogen; and

the dotted line refers to either a single bond, a double bond, or two single bonds.

In the above method, the cardiac insult can be myocardial infarction, chronic hypertension or combinations thereof

Additionally, in the above method, the compound can be administered to the subject within about 1 minute to about 16 days after the myocardial infarction.

In still yet another embodiment, the present invention relates to a method for delaying the onset of heart failure symptoms or reducing the incidence of cardiovascular events following a cardiac insult in a subject in need of treatment thereof. The method comprises the steps of:

administering to the subject in need of treatment thereof a therapeutically effective amount of at least one compound to delay the onset of heart failure symptoms or reducing the incidence of cardiovascular events following a cardiac insult, wherein said at least one compound is a xanthine oxidoreductase inhibitor or a pharmaceutically acceptable salt thereof.

In the above method, the xanthine oxidoreductase inhibitor can be selected from the group consisting of: 2-[3-cyano-4-(2-methylpropoxy)phenyl]-4-methylthiazole-5-carboxylic acid, 2-[3-cyano-4-(3-hydroxy-2-methylpropoxy)phenyl]-4-methyl-5-thiazolecarboxylic acid, 2-[3-cyano-4-(2-hydroxy-2-methylpropoxy)phenyl]-4-methyl-5-thiazolecarboxylic acid, 2-(3-cyano-4-hydroxyphenyl)-4-methyl-5-thiazolecarboxylic acid, 2-[4-(2-carboxypropoxy)-3-cyanophenyl]-4-methyl-5-thiazolecarboxylic acid, 1-(3-cyano-4-(2,2-dimethylpropoxy)phenyl)-1H-pyrazole-4-carboxylic acid, 1-3-cyano-4-(2,2-dimethylpropoxy)phenyl]-1H-pyrazole-4-carboxylic acid, pyrazolo [1,5-a]-1,3,5-triazin-4-(1H)-one, 8-[3-methoxy-4-(phenylsulfinyl)phenyl]-sodium salt (±), 3-(2-methyl-4-pyridyl)-5-cyano-4-isobutoxyphenyl)-1,2,4-triazole and a pharmaceutically acceptable salt thereof.

In the above method, the cardiac insult can be myocardial infarction, chronic hypertension or combinations thereof.

Additionally, in the above method, the compound can be administered to the subject within about 1 minute to about 16 days after the myocardial infarction.

In yet another embodiment, the present invention relates to a method for delaying the onset of heart failure symptoms or reducing the incidence of cardiovascular events following a cardiac insult in a subject in need of treatment thereof. The method comprises the steps of:

administering to the subject in need of treatment thereof a therapeutically effective amount of at least one compound to delay the onset of heart failure symptoms or reducing the incidence of cardiovascular events following a cardiac insult, wherein said compound comprises the formula:

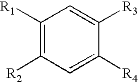

wherein R₁ and R₂ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, a phenylsulfinyl group or a cyano (—CN) group;

wherein R₃ and R₄ are each independently a hydrogen or A, B, C or D as shown below:

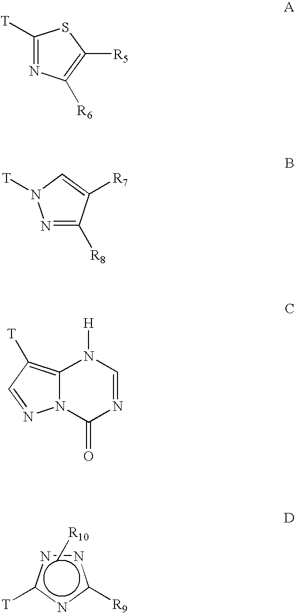

wherein T connects A, B, C or D to the aromatic ring shown above at R₁, R₂, R₃ or R₄,

wherein R₅ and R₆ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;

wherein R₇ and R₈ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;

wherein R₉ is an unsubstituted pyridyl group or a substituted pyridyl group; and

wherein R₁₀ is a hydrogen or a lower alkyl group, a lower alkyl group substituted with a pivaloyloxy group and in each case, R₁₀ bonds to one of the nitrogen atoms in the 1,2,4-triazole ring shown above.

In one aspect, the compound in the above method can be 2-[3-cyano-4-(2-methylpropoxy)phenyl]-4-methylthiazole-5-carboxylic acid or a pharmaceutically acceptable salt thereof. In another aspect, the compound in the above method can be 2-[3-cyano-4-(3-hydroxy-2-methylpropoxy)phenyl]-4-methyl-5-thiazolecarboxylic acid or a pharmaceutically acceptable salt thereof. In still yet another aspect, the compound in the above method can be 2-[3-cyano-4-(2-hydroxy-2-methylpropoxy)phenyl]-4-methyl-5-thiazolecarboxylic acid or a pharmaceutically acceptable salt thereof. In still yet another aspect, the compound in the above method can be 2-(3-cyano-4-hydroxyphenyl)-4-methyl-5-thiazolecarboxylic acid or a pharmaceutically acceptable salt thereof. In still yet another aspect, the compound in the above method can be 2-[4-(2-carboxypropoxy)-3-cyanophenyl]-4-methyl-5-thiazolecarboxylic acid or a pharmaceutically acceptable salt thereof. In still yet another aspect, the compound in the above method can be 1-3-cyano-4-(2,2-dimethylpropoxy)phenyl]-1H-pyrazole-4-carboxylic acid or a pharmaceutically acceptable salt thereof. In still yet another aspect, the compound in the above method can be pyrazolo [1,5-a]-1,3,5-triazin-4-(1H)-one, 8-[3-methoxy-4-(phenylsulfinyl)phenyl]-sodium salt (±). In still yet another aspect, the compound in the above method can be 3-(2-methyl-4-pyridyl)-5-cyano-4-isobutoxyphenyl)-1,2,4-triazole or a pharmaceutically acceptable salt thereof.

In the above method, the cardiac insult can be myocardial infarction, chronic hypertension or combinations thereof.

Additionally, in the above method, the compound can be administered to the subject within about 1 minute to about 16 days after the myocardial infarction.

In still yet another embodiment, the present invention relates to a method for delaying the onset of heart failure symptoms or reducing the incidence of cardiovascular events following a cardiac insult in a subject in need of treatment thereof. The method comprises the steps of:

administering to the subject in need of treatment thereof a therapeutically effective amount of at least one compound to delay the onset of heart failure symptoms or reducing the incidence of cardiovascular events following a cardiac insult, wherein said compound comprises the formula:

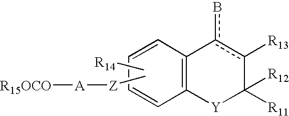

wherein R₁₁ and R₁₂ are each independently a hydrogen, a substituted or unsubstituted lower alkyl group, a substituted or unsubstituted phenyl, or R₁₁ and R₁₂ may together form a four- to eight-membered carbon ring together with the carbon atom to which they are attached;

wherein R₁₃ is a hydrogen or a substituted or unsubstituted lower alkyl group;

wherein R₁₄ is one or two radicals selected from a group consisting of a hydrogen, a halogen, a nitro group, a substituted or unsubstituted lower alkyl, a substituted or unsubstituted phenyl, —OR₁₆ and —SO₂N₁₇R_{17′}, wherein R₁₆ is a hydrogen, a substituted or unsubstituted lower alkyl, a phenyl-substituted lower alkyl, a carboxymethyl or ester thereof, a hydroxyethyl or ether thereof, or an allyl; R₁₇ and R_{17′} are each independently a hydrogen or a substituted or unsubstituted lower alkyl;

wherein R₁₅ is a hydrogen or a pharmaceutically active ester-forming group;

wherein A is a straight or branched hydrocarbon radical having one to five carbon atoms;

wherein B is a halogen, an oxygen, or an ethylenedithio;

wherein Y is an oxygen, a sulfur, a nitrogen or a substituted nitrogen;

wherein Z is an oxygen, a nitrogen or a substituted nitrogen; and

the dotted line refers to either a single bond, a double bond, or two single bonds.

In the above method, the cardiac insult can be myocardial infarction, chronic hypertension or combinations thereof.

Additionally, in the above method, the compound can be administered to the subject within about 1 minute to about 16 days after the myocardial infarction.

### BRIEF DESCRIPTION OF THE FIGURES

FIG. 1 shows that the administration of febuxostat (FBS) for 8 days, beginning ˜60 minutes after chronic transverse aortic constriction (TAC), significantly attenuated the TAC-induced left ventricular (LV) hypertrophy (p<0.01) (See, FIG. 1A) and tended to decrease the TAC-induced pulmonary congestion (See, FIG. 1B). This figure also shows that febuxostat had no effect on TAC-induced cardiac sudden death (See, FIG. 1C). Febuxostat also significantly decreased plasma levels of uric acid (See, FIG. 1D). *p<0.05 as compared with sham group; #p<0.05 as compared with vehicle group (TAC+VH); †p<0.05 versus sham group and ‡p<0.05 versus TAC+VH group using Student's paired t test.

FIG. 2 shows the administration of FBS for 8 days, beginning ˜60 minutes after chronic TAC, significantly attenuated the TAC-induced decrease of LV ejection fraction (See, FIG. 2A) and increase of LV end-diastolic posterior wall thickness (See, FIG. 2B); there was a trend toward attenuation of the increase of LV diameter at end-systole (See, FIG. 2C). However, LV diameter at end-diastole was not changed (See, FIG. 2D). *p<0.05 as compared with sham group; #p<0.05 as compared with vehicle group (TAC+VH); †p<0.05 versus sham group and ‡p<0.05 versus TAC+VH group using Student's paired t test.

FIG. 3A and FIG. 3B show the administration of FBS for 8 days, beginning ˜60 minutes after TAC, significantly attenuated the TAC-induced increase of atrial natriuretic peptide (ANP), pre-collagen-I, collagen-III, and myocardial nitrotyrosine. *p<0.05 as compared with sham group; #p<0.05 as compared with vehicle group (TAC+VH). n indicates the number of mice.

FIG. 4A and FIG. 4B show the administration of FBS for 8 days, beginning ˜60 minutes after TAC, significantly attenuated the TAC-induced increase of phosphorylation of p-Erk1 (44 KDa), p-Erk2 (42 KDa), total Erk1 (t-Erk1), total Erk2 (t-Erk2), and p-mTOR^{Ser2488}. Total Akt (t-Akt) and total mTOR (t-mTOR) were not changed after TAC. *p<0.05 as compared with sham group; #p<0.05 as compared with vehicle group (TAC+VH).

FIG. 5 shows the effects of three-week FBS or allopurinol (AL) treatment on ratios of ventricle/body and lung/body weights. Treatment was started seven days following sham or TAC procedures and continued for three weeks. * Significant difference relative to sham group. VEH=vehicle.

FIG. 6 shows the effect of FBS or AL on the survival of mice during three weeks of treatment beginning seven days following sham or TAC procedures. VEH stands for vehicle.

FIG. 7 shows the effect of three-week FBS or AL treatment on plasma uric acid. Treatment was started seven days following sham or TAC procedures and continued for three weeks. * Significant difference relative to sham group.

FIG. 8 shows the effects of three-week FBS or AL treatment on LV function and dimensions. Data are for LV ejection fraction (FIG. 8A), LV end-systolic wall thickness (FIG. 8B), LV end-systolic diameter (FIG. 8C) and LV end-diastolic diameter (FIG. 8D). Treatment was started seven days following sham or TAC procedures and continued for three weeks. * Significant difference relative to sham group. # Significant difference relative to corresponding vehicle group.

FIG. 9 shows the effect of three-week FBS treatment on cardiac myocyte diameter. FBS was administered beginning seven days following sham or TAC procedures and continued for three weeks. * Significant difference relative to sham group.

FIG. 10 shows the effects of FBS or AL on shortening fraction (FIG. 10A) and ejection fraction (FIG. 10B) in post-myocardial infarction rabbits. *p<0.05 relative to respective vehicle group; #p<0.05, mean % change from baseline is different from zero. Vehicle/Control: vehicle (0.5% methylcellulose, 2 mL/kg/day) was orally administered for 49 days starting 1 day after coronary occlusion. FBS/Early Treatment: febuxostat (FBS, 10 mg/kg/day) was orally administered for 49 days starting 1 day after coronary occlusion. FBS/Delayed Treatment: vehicle (2 mL/kg/day) was orally administered for 21 days starting 1 day after coronary occlusion followed by daily oral FBS (10 mg/kg/day) for 28 days. AL/Delayed Treatment: vehicle (2 mL/kg/day) was orally administered for 21 days starting 1 day after coronary occlusion followed by daily oral allopurinol (AL, 60 mg/kg/day) for 28 days.

FIG. 11 shows the effects of FBS or AL on left ventricular posterior wall dimension during diastole (LVPWd; FIG. 11A) and systole (LVPWs; FIG. 11B) in post-myocardial infarction rabbits. #p<0.05, mean % change from baseline is different from zero. Vehicle/Control: vehicle (0.5% methylcellulose, 2 mL/kg/day) was orally administered for 49 days starting 1 day after coronary occlusion. FBS/Early Treatment: FBS (10 mg/kg/day) was orally administered for 49 days starting 1 day after coronary occlusion. FBS/Delayed Treatment: vehicle (2 mL/kg/day) was orally administered for 21 days starting 1 day after coronary occlusion followed by daily oral FBS (10 mg/kg/day) for 28 days. AL/Delayed Treatment: vehicle (2 mL/kg/day) was orally administered for 21 days starting 1 day after coronary occlusion followed by daily oral allopurinol (AL, 60 mg/kg/day) for 28 days.

FIG. 12 shows the effects of FBS or AL on left ventricular internal dimension during diastole (LVIDd; FIG. 12A) and systole (LVIDs; FIG. 12B) in post-myocardial infarction rabbits. *p<0.05 relative to respective vehicle group; #p<0.05, mean % change from baseline is different from zero. Vehicle/Control: vehicle (0.5% methylcellulose, 2 mL/kg/day) was orally administered for 49 days starting 1 day after coronary occlusion. FBS/Early Treatment: FBS (10 mg/kg/day) was orally administered for 49 days starting 1 day after coronary occlusion. FBS/Delayed Treatment: vehicle (2 mL/kg/day) was orally administered for 21 days starting 1 day after coronary occlusion followed by daily oral FBS (10 mg/kg/day) for 28 days. AL/Delayed Treatment: vehicle (2 mL/kg/day) was orally administered for 21 days starting 1 day after coronary occlusion followed by daily oral allopurinol (AL, 60 mg/kg/day) for 28 days.

FIG. 13 shows the effects of FBS or AL on left ventricular end diastolic volume in post-myocardial infarction rabbits. #p<0.05, mean % change from baseline is different from zero. Vehicle/Control: vehicle (0.5% methylcellulose, 2 mL/kg/day) was orally administered for 49 days starting 1 day after coronary occlusion. FBS/Early Treatment: FBS (10 mg/kg/day) was orally administered for 49 days starting 1 day after coronary occlusion. FBS/Delayed Treatment: vehicle (2 mL/kg/day) was orally administered for 21 days starting 1 day after coronary occlusion followed by daily oral FBS (10 mg/kg/day) for 28 days. AL/Delayed Treatment: vehicle (2 mL/kg/day) was orally administered for 21 days starting 1 day after coronary occlusion followed by daily oral allopurinol (AL, 60 mg/kg/day) for 28 days.

FIG. 14 shows the effects of FBS or AL on left ventricular infarct size in post-myocardial infarction rabbits. Vehicle/Control: vehicle (0.5% methylcellulose, 2 mL/kg/day) was orally administered for 49 days starting 1 day after coronary occlusion. FBS/Early Treatment: FBS (10 mg/kg/day) was orally administered for 49 days starting 1 day after coronary occlusion. FBS/Delayed Treatment: vehicle (2 mL/kg/day) was orally administered for 21 days starting 1 day after coronary occlusion followed by daily oral FBS (10 mg/kg/day) for 28 days. AL/Delayed Treatment: vehicle (2 mL/kg/day) was orally administered for 21 days starting 1 day after coronary occlusion followed by daily oral allopurinol (AL, 60 mg/kg/day) for 28 days.

### DETAILED DESCRIPTION OF THE INVENTION

Definitions

Section headings as used in this section and the entire disclosure herein are not intended to be limiting.

As used herein, the singular forms “a,” “an” and “the” include plural referents unless the context clearly dictates otherwise. For the recitation of numeric ranges herein, each intervening number there between with the same degree of precision is explicitly contemplated. For example, for the range 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9 and 7.0 are explicitly contemplated.

The terms “administer”, “administering”, “administered” or “administration” refer to any manner of providing a drug (such as, a xanthine oxidoreductase inhibitor or a pharmaceutically acceptable salt thereof) to a subject or patient. Routes of administration can be accomplished through any means known by those skilled in the art. Such means include, but are not limited to, oral, buccal, intravenous, subcutaneous, intramuscular, transdermal, by inhalation and the like.

As used herein, the phrase “cardiac insult” refers to the mechanical, chemical or electrical impairment or damage of cardiac tissue due to conditions such as coronary artery disease, myocardial ischemia, myocardial infarction, myocardial inflammation, hypertension (including chronic hypertension), valvular heart disease, ventricular tachycardia, supraventricular tachycardia, imbalance of autonomic tone or the like. After experiencing or following a cardiac insult (for example, a myocardial infarction), a subject can experience at least one of cardiac hypertrophy, cardiac remodeling or left ventricular dysfunction. More specifically, the subject can experience at least two of cardiac hypertrophy, cardiac remodeling or left ventricular dysfunction. Still even more specifically, the subject can experience cardiac hypertrophy, cardiac remodeling and left ventricular dysfunction.

As used herein, the phrase “cardiac hypertrophy” refers to cardiac enlargement, a condition characterized by an increase in the size of heart and the individual cardiac muscle cells, particularly ventricular muscle cells, and an increase in the size of the inside cavity of a chamber of the heart.

As used herein, the phrase, “cardiac remodeling” refers to a compensatory physiologic response to an event or condition that compromises cardiac function. Triggers for cardiac remodeling include myocardial infarction, hypertension, wall stress, inflammation, pressure overload, and volume overload. Alterations in myocardial structure can occur as quickly as within a few hours of injury, and may progress over months and years. Thus, the phrase “cardiac remodeling” encompasses the global, cellular, and genetic changes that lead to alterations in the ventricular shape and function. While initially beneficial, these changes over time (months to years) can impair myocardial function to the point of chronic intractable heart failure. The hallmarks of cardiac remodeling are manifested as chamber dilation, increase in ventricular sphericity, and the development of interstitial and perivascular fibrosis. Increased sphericity is positively associated with mitral regurgitation. Ventricular dilation mainly results from cardiomyocyte hypertrophy and lengthening, and to a lesser extent from increases in the ventricular mass.

As used herein, the phrase “cardiovascular event(s)” refers to at least one of: a non-fatal myocardial infarction, unstable angina, a stroke, death resulting from a cardiac cause (for example, heart failure, hypertension, coronary artery disease, cardiomyopathy, a fatal myocardial infarction, etc.) or any combinations thereof that has or can occur in a subject.

As used herein, the phrase “chronic hypertension” refers to a subject which exhibits hypertension either continuously or intermittently for an extended period of time, such as, but not limited to at least one week, at least two weeks, at least three weeks, at least four weeks, at least two months, at least 6 months, at least one year, at least two years, at least three years, at least four years, at least five years, at least 10 years, etc.

As used herein, the phrase, “delaying the onset of heart failure symptoms” refers to minimizing, slowing or partially inhibiting or preventing the development of heart failure symptoms in a subject at risk for developing heart failure and need of treatment thereof. An example of a subject at risk for heart failure is a subject classified as being at Stage A or Stage B according to the stages described in Hunt, S., “ACC/AHA 2005 Guideline Update for the Diagnosis and Management of Chronic Heart Failure in the Adult: A Report of the American College of Cardiology/American Heart. Association Task Force on Practice Guidelines (Writing Committee to Update the 2001 Guidelines for the Evaluation and Management of Heart Failure),” J. Am. Coll. Cardiol., 46:e1-e82 (2005), the contents of which are herein incorporated by reference. Delaying the onset of heart failure symptoms can be assessed or determined by comparing the number of patients with newly onset of HF symptoms over or during the course of a period of time between two groups of subjects, in which a first group (the treatment group) is treated by the methods of the present invention, and a second group (the placebo group) is treated by using a placebo (namely, dummy pills) in replacement or in lieu of the treatment by the methods of the present invention. If the number of patients with newly onset of HF symptoms for the treatment group is less than the number of patients with newly onset of HF symptoms for the placebo group, then a determination is made that there was or has been delaying the onset of heart failure symptoms. However, if the number of patients with newly onset of HF symptoms for the treatment group is the same or greater than the number of patients with newly onset of HF symptoms for the placebo group, then a determination is made that there was no delaying the onset of heart failure symptoms.

As discussed previously herein, the development of HF can be characterized by considering 4 stages of the disease. The first stage, Stage A, is a subject at high risk for HF but without structural heart disease or symptoms of HF (for example, these are patients with hypertension (including chronic hypertension), atherosclerotic disease, diabetes, obesity, metabolic syndrome or patients using cardiotoxins). The second stage, Stage B, is a subject having structural heart disease but without signs or HF symptoms (for example, these are patients who have previously had a myocardial infarction, exhibit LV remodeling including LV hypertrophy and low EF, and patients with asymptomatic valvular disease). The third stage, Stage C, is a subject having structural heart disease with prior or current HF symptoms (for example, these are patients who have known structural heart disease and exhibit shortness of breath, fatigue and have reduced exercise tolerance). The fourth and final stage, Stage D, is refractory HF requiring specialized interventions (for example, patients who have marked symptoms at rest despite maximal medical therapy (namely, those who are recurrently hospitalized or cannot be safely discharged from the hospital without specialized interventions). Each of these Stages A-D is and the therapy for each of stage is described in Hunt, S., “ACC/AHA 2005 Guideline Update for the Diagnosis and Management of Chronic Heart Failure in the Adult: A Report of the American College of Cardiology/American Heart Association Task Force on Practice Guidelines (Writing Committee to Update the 2001 Guidelines for the Evaluation and Management of Heart Failure),” J. Am. Cardiol., 46:e1-e82 (2005).

As used herein, the phrase “heart failure”, refers to any condition that can result from any structural or functional cardiac disorder that impairs the ability of the heart (e.g., the ventricle) to fill with or eject blood.

As used herein, the phrase “heart failure symptoms” refers to a subject that is suffering from heart disease and is experiencing at least one of dyspnea (shortness of breath), fatigue (which may limit exercise tolerance), fluid retention (which may lead to pulmonary congestion and peripheral edema) or any combinations thereof.

As used herein, the term “hypertension” or phrase “high blood pressure” refers to a resting blood pressure, as measured with a sphygmomanometer, of greater than 120 mmHg (systolic)/80 mmHg (diastolic). Blood pressure between 121-139/81-89 is considered prehypertension and above this level (140/90 mm Hg or higher) is considered high (hypertension). Both prehypertension and hypertension blood pressure are included in the meaning of “hypertension” as used herein. For example, resting blood pressures of 135 mmHg/87 or of 140 mmHg/90 mmHg are intended to be within the scope of the term “hypertension” even though the 135/87 is within a prehypertensive category. Blood pressures of 145 mm Hg/90 mmHg, 140 mmHg/95 mmHg, and 142 mmHg/93 mmHg are further examples of high blood pressures. It will be appreciated that blood pressure normally varies throughout the day. It can even vary slightly with each heartbeat. Normally, it increases during activity and decreases at rest. It's often higher in cold weather and can rise when under stress. More accurate blood pressure readings can be obtained by daily monitoring blood pressure, where the blood pressure reading is taken at the same time each day to minimize the effect that external factors. Several readings over time may be needed to determine whether blood pressure is high.

As used herein, the phrase “left ventricular dysfunction” refers to a condition in which the left ventricle of the heart is functionally impaired. This impairment of the left ventricular causes the heart to become less efficient in pumping blood throughout the body. Left ventricular dysfunction usually leads to heart failure as well as to other cardiovascular complications. A diagnoses of left ventricular dysfunction can be made by measuring the diminished ejection fraction, mitral valve inflow pattern, pulmonary venous inflow pattern or mitral annular velocity using 2-dimensional echocardiogram coupled with Doppler flow or by performing radionuclide ventriculography, a MRI or a computed tomography using routine techniques known to those skilled the art.

As used herein, the phrase “myocardial infarction” refers to loss of cardiac myocytes or myocardial cell death caused by prolonged ischemia. Thus, ischemia is the results of obstructed blood flow to the issue.

As used herein, the phrase “pharmaceutically acceptable” includes moieties or compounds that are, within the scope of sound medical judgment, suitable for use in contact with the tissues of humans and lower animals without undue toxicity, irritation, allergic response, and the like, and are commensurate with a reasonable benefit/risk ratio.

As used herein, the phrase “reducing the incidences of cardiovascular events” means maintaining or reducing the number of non-fatal cardiovascular events experienced by a subject during or over the course of a period of time. A reduction in the incidence of cardiovascular events can be assessed or determined by comparing the incidences of cardiovascular events over or during the course of a period of time between two groups of subjects, in which a first group (the treatment group) is treated by the methods of the present invention, and a second group (the placebo group) is treated by using a placebo (namely, dummy pills) in replacement or in lieu of the treatment by the methods of the present invention. If the number of cardiovascular events for the treatment group is less than the number of the cardiovascular events for the placebo group, then a determination is made that there was or has been a reduction in the incidences of cardiovascular events. However, if the number of cardiovascular events for the treatment group is the same or greater than the number of cardiovascular events for the placebo group, then a determination is made that there was no reduction in the incidences of cardiovascular events. Alternatively, a reduction in the incidence of non-fatal cardiovascular events can be assessed or determined by determining a baseline number of non-fatal cardiovascular events for a subject at a first period in time and then measuring the number of non-fatal cardiovascular events for a subject at a second, later period in time. If the number of non-fatal cardiovascular events for the subject at the second, later period in time is the same as or less then the number of non-fatal cardiovascular events for the subject at the first period in time, then a determination is made that there has been a reduction in the incidences of cardiovascular events for said subject. However, if the number of non-fatal cardiovascular events for a subjected at the second, later period in time is greater then the number of non-fatal cardiovascular events for the subject at the first period in time, then a determination is made that there was not a reduction in the incidences of cardiovascular events for the subject, but rather an increase in incidences of cardiovascular events for the subject.

As used herein, the phrase “reducing the progression of cardiac hypertrophy” means maintaining the size of heart or the size of the inside cavity of a chamber of the heart. A reduction in the progression of cardiac hypertrophy can be assessed by comparing the size of heart or the size of the inside cavity of a chamber of the heart over or during the course of a period of time between two groups of subjects, in which a first group (the treatment group) is treated by the methods of the present invention, and a second group (the placebo group) is treated by using a placebo (namely, dummy pills) in replacement or in lieu of the treatment by the methods of the present invention. If the size of heart or the size of the inside cavity of a chamber of the heart for the treatment group is less than the size of heart or the size of the inside cavity of a chamber of the heart for the placebo group, then a determination is made that there was or has been a reduction in the progression of cardiac hypertrophy. However, if the size of heart or the size of the inside cavity of a chamber of the heart for treatment group is the same or greater than the size of heart or the size of the inside cavity of a chamber of the heart for the placebo group, then a determination is made that there was no reduction in the progression of cardiac hypertrophy. Methods for determining disease progression or development, such as cardiac hypertrophy, can be assessed using well known methods, such as physical examination, a 2-dimensional echocardiogram coupled with Doppler flow studies, ultrasound, a MRI, computerized tomography, cardiac catheterization, radionuclide imaging (such as radionuclide ventriculography) any combinations thereof.

As used herein, the phrase “reducing the progression of cardiac remodeling” means maintaining myocardial structure or decreasing the alterations in myocardial structure of the heart in a subject. A reduction in the progression of cardiac remodeling can be assessed by comparing the alterations in myocardial structure of the heart over a period of time between two groups of subjects, in which a first group (the treatment group) is treated by the methods of the present invention, and a second group (the placebo group) is treated by using a placebo (namely, dummy pills) in replacement or in lieu of the treatment by the methods of the present invention. If the alterations in myocardial structure of the heart in the subjects of the treatment group are less than the alterations in myocardial structure of the heart in the subjects of the placebo group, then a determination is made that there has been a reduction in the progression of cardiac remodeling. However, if the alterations in myocardial structure of the heart in the subjects of the treatment group are the same or more than the alterations in myocardial structure of the heart in the subjects of the placebo group, then a determination is made that there was not a reduction in the progression of cardiac remodeling. Methods for determining disease progression or development, such as cardiac remodeling, can be assessed using well known methods, such as physical examination, a 2-dimensional echocardiogram coupled with Doppler flow studies, ultrasound, a MRI, computerized tomography, cardiac catheterization, radionuclide imaging (such as radionuclide ventriculography) and any combinations thereof.

As used herein, the phrase “reducing the progression of left ventricular dysfunction” means maintaining or improving the efficiency of the heart to pump blood throughout the body of a subject. A reduction in the progression of left ventricular dysfunction can be assessed by comparing the efficiency of the heart to pump blood throughout the body of a subject over or during the course of a period of time between two groups of subjects, in which a first group (the treatment group) is treated by the methods of the present invention, and a second group (the placebo group) is treated by using a placebo (the dummy pills) in replacement or in lieu of the treatment by the methods of the present invention. If the efficiency of the heart to pump blood throughout the body of a subject in the treatment group is better than the efficiency of the heart to pump blood throughout the body of a subject in the placebo group, then a determination is made that there has been a reduction in the progression of left ventricular dysfunction. However, if the efficiency of the heart to pump blood throughout the body of a subject in treatment group are the same or worse than the efficiency of the heart to pump blood throughout the body of a subject in the placebo group, then a determination is made that there was no reduction in the progression of left ventricular dysfunction. Alternatively, a reduction in the progression of left ventricular dysfunction can be determined by measuring at least one of left ventricular ejection fraction, the motility of the left ventricular wall or combinations thereof. Methods for determining disease progression or development, such as left ventricular dysfunction, can be assessed using well known methods, such as physical examination, a 2-dimensional echocardiogram coupled with Doppler flow studies, ultrasound, a MRI, computerized tomography, cardiac catheterization, radionuclide imaging (such as radionuclide ventriculography), hemodynamic monitoring magnetic resonance angiography, exercise treadmill testing coupled with oxygen uptake studies and any combinations thereof.

As used herein, the term “subject” refers to an animal, preferably a mammal, including a human or non-human. The terms patient and subject may be used interchangeably herein.

The phrases “therapeutically effective amount” or “prophylactically effective amount” of a drug (namely, at least one xanthine oxidoreductase inhibitor or a salt thereof) refers to a nontoxic but sufficient amount of the drug to provide the desired effect. The desired effect may be one or more of reducing the progression of at least one of cardiac hypertrophy, cardiac remodeling or left ventricular dysfunction following a cardiac insult in a subject in need of treatment thereof or delaying the onset of heart failure symptoms or reducing the incidence of cardiovascular events following a cardiac insult in a subject in need of treatment thereof. As with other pharmaceuticals, it will be understood that the total daily usage of a pharmaceutical composition of the invention will be decided by a patient's attending physician within the scope of sound medical judgment. The specific therapeutically effective or prophylactically effective dose level for any particular patient will depend upon a variety of factors including the disorder being treated and the severity of the disorder; activity of the specific compound employed; the specific composition employed; the age, body weight, general health, sex and diet of the patient; the time administration, route of administration, and rate of excretion of the specific compound employed; the duration of the treatment; drugs used in combination or coincidental with the specific compound employed; and other factors known to those of ordinary skill in the medical arts. For example, it is well within the skill of the art to start doses of the compound at levels lower than required to achieve the desired therapeutic effect and to gradually increase the dosage until the desired effect is achieved.

Accordingly, the amount of drug that is “effective” or “prophylactic” will vary from subject to subject, depending on the age and general condition of the individual, the particular drug or drugs, and the like. Thus, it is not always possible to specify an exact “therapeutically effective amount” or a “prophylactically effective amount”. However, an appropriate “therapeutically effective amount” or “prophylactically effective amount” in any individual case may be determined by one skilled in the art.

The terms “treating” and “treatment” refer to reduction in severity and/or frequency of symptoms, elimination of symptoms and/or underlying cause, prevention of the occurrence of symptoms and/or their underlying cause, and improvement or remediation of damage. Thus, for example, “treating” a patient involves prevention of a particular disorder or adverse physiological event in a susceptible individual as well as treatment of a clinically symptomatic individual by inhibiting or causing regression of a disorder or disease.

As used herein, the phrase “xanthine oxidoreductase inhibitor” refers to any compound that (1) is an inhibitor of a xanthine oxidoreductase, such as, but not limited to, xanthine oxidase; and (2) chemically, does not contain a purine ring in its structure (i.e. is a “non-purine”). The phrase “xanthine oxidoreductase inhibitor” as defined herein also includes metabolites, polymorphs, solvates and prodrugs of the such compounds, including metabolites, polymorphs, solvates and prodrugs of the exemplary compounds described as Formula I and Formula II below. Examples of xanthine oxidoreductase inhibitors include, but are not limited to, 2-[4-(2-carboxypropoxy)-3-cyanophenyl]-4-methyl-5-thiazolecarboxylic acid and compounds having the following Formula I or Formula II:

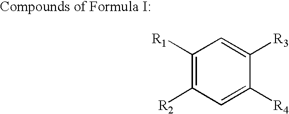

wherein R₁ and R₂ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, a phenylsulfinyl group or a cyano (—CN) group; wherein R₃ and R₄ are each independently a hydrogen or A, B, C or D as shown below:

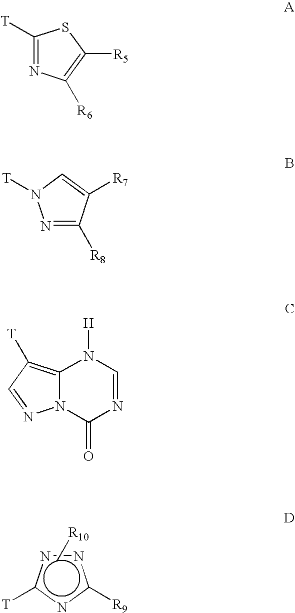

wherein T connects or attaches A, B, C or D to the aromatic ring shown above at R₁, R₂, R₃ or R₄.

wherein R₅ and R₆ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;

wherein R₇ and R₈ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;

wherein R₉ is an unsubstituted pyridyl group or a substituted pyridyl group; and

wherein R₁₀ is a hydrogen or a lower alkyl group, a lower alkyl group substituted with a pivaloyloxy group and in each case, R₁₀ bonds to one of the nitrogen atoms in the 1,2,4-triazole ring shown above in Formula I.

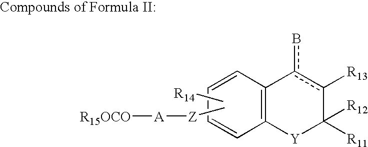

wherein R₁₁ and R₁₂ are each independently a hydrogen, a substituted or unsubstituted lower alkyl group, a substituted or unsubstituted phenyl (the substituted phenyl in this Formula II refers to a phenyl substituted with a halogen or lower alkyl, and the like. Examples include, but are not limited to, p-tolyl and p-chlorophenyl), or R₁₁ and R₁₂ may together form a four- to eight-membered carbon ring together with the carbon atom to which they are attached;

wherein R₁₃ is a hydrogen or a substituted or unsubstituted lower alkyl group;

wherein R₁₄ is one or two radicals selected from a group consisting of a hydrogen, a halogen, a nitro group, a substituted or unsubstituted lower alkyl group, a substituted or unsubstituted phenyl (the substituted phenyl in this Formula II refers to a phenyl substituted with a halogen or lower alkyl group, and the like. Examples include, but are not limited to, p-tolyl and p-chlorophenyl), —OR₁₆ and —SO₂NR₁₇R_{17′}, wherein R₁₆ is a hydrogen, a substituted or unsubstituted lower alkyl, a phenyl-substituted lower alkyl, a carboxymethyl or ester thereof, a hydroxyethyl or ether thereof, or an allyl; R₁₇ and R_{17′} are each independently a hydrogen or a substituted or unsubstituted lower alkyl group;

wherein R₁₅ is a hydrogen or a pharmaceutically active ester-forming group;

wherein A is a straight or branched hydrocarbon radical having one to five carbon atoms;

wherein B is a halogen, an oxygen, or an ethylenedithio;

wherein Y is an oxygen, a sulfur, a nitrogen or a substituted nitrogen;

wherein Z is an oxygen, a nitrogen or a substituted nitrogen; and

the dotted line refers to either a single bond, a double bond, or two single bonds (for example, when B is ethylenedithio, the dotted line shown in the ring structure can be two single bonds).

As used herein, the term “lower alkyl(s)” group refers to a C₁-C₇ alkyl group, including, but not limited to, including methyl, ethyl, n-propyl, isopropyl, butyl, isobutyl, sec-butyl, tert-butyl, pentyl, isopentyl, hexyl, heptal and the like.

As used herein, the term “lower alkoxy” refers to those groups formed by the bonding of a lower alkyl group to an oxygen atom, including, but not limited to, methoxy, ethoxy, propoxy, isopropoxy, butoxy, isobutoxy, pentoxy, hexoxy, heptoxy and the like.

As used herein, the term “lower alkylthio group” refers to those groups formed by the bonding of a lower alkyl to a sulfur atom.

As used herein, the term “halogen” refers to fluorine, chlorine, bromine and iodine.

As used herein, the term “substituted pyridyl” refers to a pyridyl group that can be substituted with a halogen, a cyano group, a lower alkyl, a lower alkoxy or a lower alkylthio group.

As used herein, the term “four- to eight-membered carbon ring” refers to cyclobutyl, cyclopentyl, cyclohexyl, cycloheptyl, cyclooctyl and the like.

As used herein, the phrase “pharmaceutically active ester-forming group” refers to a group which binds to a carboxyl group through an ester bond. Such ester-forming groups can be selected from carboxy-protecting groups commonly used for the preparation of pharmaceutically active substances, especially prodrugs. For the purpose of the present invention, said group should be selected from those capable of binding to compounds having Formula II wherein R₁₅ is hydrogen through an ester bond. Resultant esters are effective to increase the stability, solubility, and absorption in gastrointestinal tract of the corresponding non-esterified forms of said compounds having Formula II, and also prolong the effective blood-level of it. Additionally, the ester bond can be cleaved easily at the pH of body fluid or by enzymatic actions in vivo to provide a biologically active form of the compound having Formula II. Preferred pharmaceutically active ester-forming groups include, but are not limited to, 1-(oxygen substituted)-C₂ to C₁₅ alkyl groups, for example, a straight, branched, ringed, or partially ringed alkanoyloxyalkyl groups, such as acetoxymethyl, acetoxyethyl, propionyloxymethyl, pivaloyloxymethyl, pivaloyloxyethyl, cyclohexaneacetoxyethyl, cyclohexanecarbonyloxycyclohexylmethyl, and the like, C₃ to C₁₅ alkoxycarbonyloxyalkyl groups, such as ethoxycarbonyloxyethyl, isopropoxycarbonyloxyethyl, isopropoxycarbonyloxypropyl, t-butoxycarbonyloxyethyl, isopentyloxycarbonyloxypropyl, cyclohexyloxycarbonyloxyethyl, cyclohexylmethoxycarbonyloxyethyl, bornyloxycarbonyloxyisopropyl, and the like, C₂ to C₈ alkoxyalkyls, such as methoxy methyl, methoxy ethyl, and the like, C₄ to C₈ 2-oxacycloalkyls such as, tetrahydropyranyl, tetrahydrofuranyl, and the like, substituted C₈ to C₁₂ aralkyls, for example, phenacyl, phthalidyl, and the like, C₆ to C₁₂ aryl, for example, phenyl xylyl, indanyl, and the like, C₂ to C₁₂ alkenyl, for example, allyl, (2-oxo-1,3-dioxolyl)methyl, and the like, and [4,5-dihydro-4-oxo-1H-pyrazolo[3,4-d]pyrimidin-1-yl]methyl, and the like.

In R₁₆ in Formula II, the term “ester” as used in the phrase “the ester of carboxymethyl” refers to a lower alkyl ester, such as methyl or ethyl ester; and the term “ether” used in the phrase “the ether of hydroxyethyl” means an ether which is formed by substitution of the hydrogen atom of hydroxyl group in the hydroxyethyl group by aliphatic or aromatic alkyl group, such as benzyl.

The carboxy-protecting groups may be substituted in various ways. Examples of substituents include halogen atom, alkyl groups, alkoxy groups, alkylthio groups and carboxy groups.

As used herein, the term “straight or branched hydrocarbon radical” in the definition of A in Formula II above refers to methylene, ethylene, propylene, methylmethylene, or isopropylene.

As used herein, the substituent of the “substituted nitrogen” in the definition of Y and Z in Formula II above are hydrogen, lower alkyl, or acyl.

As used herein, the term “phenyl-substituted lower alkyl” refers to a lower alkyl group substituted with phenyl, such as benzyl, phenethyl or phenylpropyl.

As used herein, the term “prodrug” refers to a derivative of the compounds shown in the above-described Formula I and Formula II that have chemically or metabolically cleavable groups and become by solvolysis or under physiological conditions compounds that are pharmaceutically active in vivo. Esters of carboxylic acids are an example of prodrugs that can be used in the dosage forms of the present invention. Methyl ester prodrugs may be prepared by reaction of a compound having the above-described formula in a medium such as methanol with an acid or base esterification catalyst (e.g., NaOH, H₂SO₄). Ethyl ester prodrugs are prepared in similar fashion using ethanol in place of methanol.

Examples of compounds having the above Formula I are: 2-[3-cyano-4-(2-methylpropoxy)phenyl]-4-methylthiazole-5-carboxylic acid (also known as “febuxostat”), 2-[3-cyano-4-(3-hydroxy-2-methylpropoxy)phenyl]-4-methyl-5-thiazolecarboxylic acid, 2-[3-cyano-4-(2-hydroxy-2-methylpropoxy)phenyl]-4-methyl-5-thiazolecarboxylic acid, 2-(3-cyano-4-hydroxyphenyl)-4-methyl-5-thiazolecarboxylic acid, 2-[4-(2-carboxypropoxy)-3-cyanophenyl]-4-methyl-5-thiazolecarboxylic acid, 1-(3-cyano-4-(2,2-dimethylpropoxy)phenyl)-1H-pyrazole-4-carboxylic acid, 1-3-cyano-4-(2,2-dimethylpropoxy)phenyl]-1H-pyrazole-4-carboxylic acid, pyrazolo [1,5-a]-1,3,5-triazin-4-(1H)-one, 8-[3-methoxy-4-(phenylsulfinyl)phenyl]-sodium salt (±) or 3-(2-methyl-4-pyridyl)-5-cyano-4-isobutoxyphenyl)-1,2,4-triazole.

Preferred compounds having the above Formula I are: 2-[3-cyano-4-(2-methylpropoxy)phenyl]-4-methylthiazole-5-carboxylic acid, 2-[3-cyano-4-(3-hydroxy-2-methylpropoxy)phenyl]-4-methyl-5-thiazolecarboxylic acid, 2-[3-cyano-4-(2-hydroxy-2-methylpropoxy)phenyl]-4-methyl-5-thiazolecarboxylic acid, 2-(3-cyano-4-hydroxyphenyl)-4-methyl-5-thiazolecarboxylic acid, 2-[4-(2-carboxypropoxy)-3-cyanophenyl]-4-methyl-5-thiazolecarboxylic acid. These preferred compounds have also been found not have an effect at a therapeutically effective amount in a subject on the activity of any of the following enzymes involved in purine and pyrimidine metabolism: guanine deaminase, hypoxanthine-guanine phosphoribosyltransferse, purine nucleotide phosphorylase, orotate phosphoribosyltransferase or orotidine-5-monophosphate decarboxylase (i.e., meaning that it is “selective” for none of these enzymes which are involved in purine and pyrimidine metabolism). Assays for determining the activity for each of the above-described enzymes is described in Yasuhiro Takano, et al., Life Sciences, 76:1835-1847 (2005).

These preferred compounds have also been referred to in the literature as nonpurine, selective inhibitors of xathine oxidase (NP/SIXO).

Examples of compounds having the above Formula II are described in U.S. Pat. No. 5,268,386 and EP 0 415 566 A1.

With the exception of pyrazolo [1,5-a]-1,3,5-triazin-4-(1H)-one, 8-[3-methoxy-4-(phenylsulfinyl)phenyl]-sodium salt (±), methods for making xanthine oxidoreductase inhibiting compounds of Formulas I and II for use in the methods of the present invention are known in the art and are described, for example, in U.S. Pat. Nos. 5,268,386, 5,614,520, 6,225,474, 7,074,816 and EP 0 415 566 A1 and in the publications Ishibuchi, S. et al., Bioorg. Med. Chem. Lett., 11:879-882 (2001) and which are each herein incorporated by reference. Other xanthine oxidoreductase inhibiting compounds can be found using xanthine oxidoreductase and xanthine in assays to determine if such candidate compounds inhibit conversion of xanthine into uric acid. Such assays are well known in the art.

Pyrazolo [1,5-a]-1,3,5-triazin-4-(1H)-one, 8-[3-methoxy-4-(phenylsulfinyl)phenyl]-sodium salt (±) is available from Otsuka Pharmaceutical Co. Ltd. (Tokyo, Japan) and is described in the following publications: Uematsu T., et al., “Pharmacokinetic and Pharmacodynamic Properties of a Novel Xanthine Oxidase Inhibitor, BOF-4272, in Healthy Volunteers, J. Pharmacology and Experimental Therapeutics, 270:453-459 (August 1994), Sato, S., A Novel Xanthine Deydrogenase Inhibitor (BOF-4272). In Purine and Pyrimidine Metabolism in Man, Vol. VII, Part A, ed. By P. A. Harkness, pp. 135-138, Plenum Press, New York. Pyrazolo [1,5-a]-1,3,5-triazin-4-(1H)-one, 8-[3-methoxy-4-(phenylsulfinyl)phenyl]-sodium salt (±) can be made using routine techniques known in the art.

### The Invention

In one embodiment, the present invention relates to methods of reducing the progression of at least one of cardiac hypertrophy, cardiac remodeling or left ventricular dysfunction in a subject following a cardiac insult and whose is in need of treatment thereof. The methods according to this embodiment of the present invention involve administering to a subject following a cardiac insult and in need of treatment thereof a therapeutically effective amount of at least one xanthine oxidoreductase inhibitor or a pharmaceutically acceptable salt thereof to delay or reduce the progression of at least one of cardiac hypertrophy, cardiac remodeling or left ventricular dysfunction in that subject.

An example of a subject in need of treatment thereof that can benefit from the methods of the present invention are subjects that have been classified as being in Stage A or B pursuant to stages described in Hunt, S., “ACC/AHA 2005 Guideline Update for the Diagnosis and Management of Chronic Heart Failure in the Adult: A Report of the American College of Cardiology/American Heart Association Task Force on Practice Guidelines (Writing Committee to Update the 2001 Guidelines for the Evaluation and Management of Heart Failure),” J. Am. Coll. Cardiol., 46:e1-e82 (2005). For example, a subject in Stage A might be suffering from hypertension (including chronic hypertension), and a subject in Stage B might had suffered a myocardial infarction.

It has also been found that for those subjects who have suffered cardiac insult that the timing of administration of the at least one xanthine oxidoreductase inhibitor according to the methods of the present invention can provide useful benefits and results. Specifically, it has been discovered that the administration to a subject of at least one xanthine oxidoreductase inhibitor according to the methods of the present invention within about 1 minute to about 16 days, more specifically within about 5 minutes to about 10 days, even more specifically, within about 10 minutes to about 5 days after the subject has suffered a myocardial infarction is particularly useful in delaying or reducing the progression of at least one of cardiac hypertrophy, cardiac remodeling or left ventricular dysfunction in the subject.

Routine techniques known to those skilled in the art can be used to determine whether there is reduction or delay in the progression of at least one of cardiac hypertrophy, cardiac remodeling or left ventricular dysfunction in a subject following a cardiac insult that is being treated pursuant to the methods of the present invention. For example, as discussed previously herein, a reduction or delay in the progression of cardiac hypertrophy can be determined by comparing the size of heart or the size of the inside cavity of a chamber of the heart over a period of time between two groups of subjects, in which a first group (the treatment group) is treated by the methods of the first embodiment of the present invention, and a second group (the placebo group) is treated by using a placebo (namely, dummy pills) in replacement or in lieu of the treatment by the methods of the first embodiment of the present invention. If the size of heart or the size of the inside cavity of a chamber of the heart for the treatment group is less than the size of heart or the size of the inside cavity of a chamber of the heart for the placebo group, then a determination is made that there was or has been a reduction in the progression of cardiac hypertrophy. However, if the size of heart or the size of the inside cavity of a chamber of the heart for treatment group is the same or greater than the size of heart or the size of the inside cavity of a chamber of the heart for the placebo group, then a determination is made that there was no reduction in the progression of cardiac hypertrophy.

The methods of this embodiment of the present invention can be used solely as a mono therapy. Alternatively, the methods of this embodiment of the present invention can be used as part of a combination therapy. For example, the methods of the present invention can be used as a part of a combination therapy with other drugs that are known to prevent or delay the onset of HF at its various stages (namely, Stages A-D). These drugs include, but are not limited to, vasodilators, angiotensin II receptor antagonists, angiotensin converting enzyme inhibitors, aldosterone antagonists, diuretics, hydralazine/nitrates, antithrombolytic agents, β-adrenergic receptor antagonists, α-adrenergic receptor antagonists, calcium channel blockers, etc.

In a second embodiment, the present invention relates to a method of delaying the onset of heart failure symptoms or reducing the incidence of cardiovascular events following a cardiac insult in a subject in need of treatment thereof. The methods according to this embodiment of the present invention involve administering to a subject following a cardiac insult and in need of treatment thereof a therapeutically effective amount of at least one xanthine oxidoreductase inhibitor or a pharmaceutically acceptable salt thereof to delay the onset of heart failure symptoms or reduce the incidence of cardiovascular events in that subject.

An example of a subject in need of treatment thereof that would benefit from the methods of this embodiment are subjects that have been classified as being in Stage A or B pursuant to stages described in Hunt, S., “ACC/AHA 2005 Guideline Update for the Diagnosis and Management of Chronic Heart Failure in the Adult: A Report of the American College of Cardiology/American Heart Association Task Force on Practice Guidelines (Writing. Committee to Update the 2001 Guidelines for the Evaluation and Management of Heart Failure),” J. Am. Coil. Cardiol., 46:e1-e82 (2005). For example, a subject in Stage B might be suffering from hypertension (including chronic hypertension), and a subject in Stage B might had suffered a myocardial infarction.

It has also been found that for those subjects who have suffered cardiac insult that the timing of administration of the at least one xanthine oxidoreductase inhibitor according to the methods of the present invention can provide useful benefits and results. Specifically, it has been discovered that the administration to a subject of at least one xanthine oxidoreductase inhibitor according to the methods of the present invention within about 1 minute to about 16 days, more specifically within about 5 minutes to about 10 days, even more specifically, within about 10 minutes to about 5 days after the subject has suffered a myocardial infarction is particularly useful in delaying the onset of heart failure symptoms or reducing the incidence of cardiovascular events in a subject.

Routine techniques known to those skilled in the art can be used to determine whether there is a delay in the onset of heart failure symptoms or reducing the incidence of cardiovascular events in a subject that is being treated pursuant to the methods of the present invention. For example, as discussed previously herein, a reduction in the incidence of cardiovascular events can be assessed or determined by comparing the incidences of cardiovascular events over period of time between two groups of subjects, in which a first group (the treatment group) is treated by the methods of the present invention, and a second group (the placebo group) is treated by using a placebo (namely, dummy pills) in replacement or in lieu of the treatment by the methods of the present invention. If the number of cardiovascular events for the treatment group is less than the number of the cardiovascular events for the placebo group, then a determination is made that there was or has been a reduction in the incidences of cardiovascular events. However, if the number of cardiovascular events for the treatment group is the same or greater than the number of cardiovascular events for the placebo group, then a determination is made that there was no reduction in the incidences of cardiovascular events.

The methods of this second embodiment of the present invention can be used solely as a mono therapy. Alternatively, the methods of this second embodiment of the present invention can be used as part of a combination therapy. For example, the methods of the present invention can be used as a part of a combination therapy with other drugs that are known to prevent or delay the onset of HF at its various stages (namely, Stages A-D). These drugs include, but are not limited to, vasodilators, angiotensin II receptor antagonists, angiotensin converting enzyme inhibitors, aldosterone antagonists, diuretics, hydralazine/nitrates, antithrombolytic agents, β-adrenergic receptor antagonists, α-adrenergic receptor antagonists, calcium channel blockers, etc.

Compositions containing at least one xanthine oxidoreductase inhibitor are contemplated for use in the methods of the present invention. Using the excipients and dosage forms described below, formulations containing such combinations are a matter of choice for those skilled in the art. Further, those skilled in the art will recognize that various coatings or other separation techniques may be used in cases where the combination of compounds are incompatible.

Compounds for use in accordance with the methods of the present invention can be provided in the form of pharmaceutically acceptable salts derived from inorganic or organic acids. Pharmaceutically acceptable salts are well-known in the art. For example, S. M. Berge et al. describe pharmaceutically acceptable salts in detail in J. Pharmaceutical Sciences, 66: 1 et seq. (1977). The salts can be prepared in situ during the final isolation and purification of the compounds or separately by reacting a free base function with a suitable organic acid. Representative acid addition salts include, but are not limited to, acetate, adipate, alginate, citrate, aspartate, benzoate, benzenesulfonate, bisulfate, butyrate, camphorate, camphor sulfonate, digluconate, glycerophosphate, hemisulfate, heptanoate, hexanoate, fumarate, hydrochloride, hydrobromide, hydroiodide, 2-hydroxyethansulfonate(isothionate), lactate, maleate, methane sulfonate, nicotinate, 2-naphthalene sulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, basic nitrogen-containing groups can be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid.

Basic addition salts can be prepared in situ during the final isolation and purification of compounds by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylammonium, dimethylammonium, trimethylammonium, triethylammonium, diethylammonium, and ethylammonium among others. Other representative organic amines useful for the formation of base addition salts include ethylenediamine, ethanolamine, diethanolamine, piperidine, piperazine and the like.

The at least one xanthine oxidoreductase inhibiting compound or salts thereof, may be formulated in a variety of ways that is largely a matter of choice depending upon the delivery route desired. For example, solid dosage forms for oral administration include capsules, tablets, pills, powders and granules. In such solid dosage forms, the xanthine oxidoreductase inhibiting compound may be mixed with at least one inert, pharmaceutically acceptable excipient or carrier, such as sodium citrate or dicalcium phosphate and/or a) fillers or extenders, such as, but not limited to, starches, lactose, sucrose, glucose, mannitol and silicic acid; b) binders, such as, but not limited to, carboxymethylcellulose, alginates, gelatin, polyvinylpyrrolidone, sucrose and acacia; c) humectants, such as, but not limited to glycerol; d) disintegrating agents, such as, but not limited to, agar-agar, calcium carbonate, potato or tapioca starch, alginic acid, certain silicates and sodium carbonate; e) solution retarding agents, such as, but not limited to, paraffin; f) absorption accelerators, such as, but not limited to, quaternary ammonium compounds; g) wetting agents, such as, but not limited to, cetyl alcohol and glycerol monostearate; h) absorbents, such as, but not limited to, kaolin and bentonite clay; and i) lubricants, such as, but not limited to, talc, calcium stearate, magnesium stearate, solid polyethylene glycols, sodium lauryl sulfate and mixtures thereof.

Solid compositions of a similar type may also be employed as fillers in soft and hard-filled gelatin capsules using such excipients as lactose or milk sugar as well as high molecular weight polyethylene glycols and the like.

The solid dosage forms of tablets, capsules, pills and granules can be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions which can be used include polymeric substances and waxes.

Liquid dosage forms for oral administration include pharmaceutically acceptable emulsions, solutions, suspensions, syrups and elixirs. In addition to the xanthine oxidoreductase inhibiting compounds, the liquid dosage forms may contain inert diluents commonly used in the art such as, for example, water or other solvents, solubilizing agents and emulsifiers, such as, but not limited to, ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof.

The compositions can also be delivered through a catheter for local delivery at a target site, via an intracoronary stent (a tubular device composed of a fine wire mesh), or via a biodegradable polymer.

Compositions suitable for parenteral injection may comprise physiologically acceptable, sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include, but are not limited to, water, ethanol, polyols (propylene glycol, polyethylene glycol, glycerol, and the like), vegetable oils (such as olive oil), injectable organic esters such as ethyl oleate, and suitable mixtures thereof.

These compositions can also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example, sugars, sodium chloride and the like. Prolonged absorption of the injectable pharmaceutical form can be brought about by the use of agents delaying absorption, for example, aluminum monostearate and gelatin.

Suspensions, in addition to the active compounds (i.e., xanthine oxidoreductase inhibiting compounds or salts thereof), may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Proper fluidity can be maintained, for example, by the use of coating materials such as lecithin, by the maintenance of the required particle size in the case of dispersions and by the use of surfactants.

In some cases, in order to prolong the effect of the drug (i.e. xanthine oxidoreductase inhibiting compounds or salts thereof), it is desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Dosage forms for topical administration of the compounds of this present invention include powders, sprays, ointments and inhalants. The active compound(s) is mixed under sterile conditions with a pharmaceutically acceptable carrier and any needed preservatives, buffers or propellants which can be required. Opthalmic formulations, eye ointments, powders and solutions are also contemplated as being within the scope of this invention.

It will be understood that formulations used in accordance with the present invention generally will comprise a therapeutically effective amount of one or more xanthine oxidoreductase inhibiting compounds.

Formulations of the present invention are administered and dosed in accordance with sound medical practice, taking into account the clinical condition of the individual patient, the site and method of administration, scheduling of administration, and other factors known to medical practitioners.

Therapeutically effective or prophylactically effective amounts for purposes herein thus can readily be determined by such considerations as are known to those skilled in the art. The daily therapeutically effective or prophylactically effective amount of the xanthine oxidoreductase inhibiting compounds administered to a patient in single or divided doses range from about 0.01 to about 750 milligram per kilogram of body weight per day (mg/kg/day). More specifically, a patient may be administered from about 5.0 mg to about 300 mg once daily, preferably from about 20 mg to about 240 mg once daily and most preferably from about 40 mg to about 120 mg once daily of xanthine oxidoreductase inhibiting compounds. Of course, it will be understood by one skilled in the art that other dosage regimens may be utilized, such as dosing more than once per day, utilizing extended, sustained, controlled, or modified release dosage forms, and the like in order to achieve the desired result of reducing the progression of at least one of cardiac hypertrophy, cardiac remodeling or left ventricular dysfunction following a cardiac insult in a subject in need of treatment thereof or delaying the onset of heart failure symptoms or reducing the incidence of cardiovascular events following a cardiac insult in a subject in need of treatment thereof.

By way of example, and not of limitation, examples of the present invention will now be given.

### Example 1

### Prevention and Treatment Effects of Febuxostat on Systolic Overload-Induced Ventricular Hypertrophy and Congestive Heart Failure in Mice

### Materials and Methods

Mice. Male C57/BL6 mice at 8-9 weeks of age were purchased from Jackson Laboratories (Bar Harbor, Me.) for use in this study. Mice were housed in an air-conditioned room with a 12-hour:12-hour light-dark cycle, received standard mouse chow, and drank tap water. This study was approved by the Institutional Animal Care and Use Committee of the University of Minnesota.
Experimental Protocol 1. This protocol was designed to determine whether febuxostat (2-[3-cyano-4-(2-methylpropoxy)phenyl]-4-methylthiazole-5-carboxylic acid) treatment, beginning immediately after the onset of acute pressure overload, could protect against transverse aortic constriction (TAC)-induced left ventricular (LV) hypertrophy, dysfunction and mortality. Three treatment groups were included (See Table 1, below): sham-operated mice (n=20), TAC mice treated with vehicle (0.5% methylcellulose) (n=30), and TAC mice treated with febuxostat (suspension in 0.5% methylcellulose) (n=30). To generate LV hypertrophy in a short time period with a significant incidence of cardiac mortality, TAC was created by ligating the transverse aorta against a 27 G needle to create severe LV pressure overload. Since mice tend to take in less water and food during the first 1-3 days after the TAC procedure, febuxostat (5 mg/kg/day in 250 μl vehicle) or vehicle (250 μl) was administered daily by oral gavage for a total of 8 days, with the first dose administered on the day of the surgery after the animal awoke from anesthesia (˜60 min after the TAC procedure). On the eighth day, LV function was assessed by echocardiography approximately 1 hour after the last febuxostat or vehicle dose. Approximately 3 hours after echocardiographic evaluation (4 hours after the last febuxostat or vehicle dose), the mice were weighed and blood samples collected. The animals were euthanized and the heart and lungs excised; the right and left atria were trimmed away, and the ventricles and lungs were weighed. Tissue from the left ventricle was used for histological evaluation and Western blots.
Experimental Protocol 2. This protocol was designed to determine whether administration of febuxostat (FBS) or allopurinol (AL) beginning after the onset of cardiac hypertrophy, LV dysfunction and heart failure could exert a protective effect. For this protocol, mice subjected to TAC (using a 26 gauge (G) needle to generate moderate TAC) or sham surgery were divided into six experimental groups as described in Table 1, below. Beginning seven days after TAC, animals were treated with vehicle (0.01 mg/ml NaCl, equivalent salt content as in the FBS drinking water), FBS (0.05 mg/ml) or AL (0.15 mg/ml) in drinking water for three weeks. Following three weeks of treatment, ventricular function was assessed by echocardiography. Blood samples and cardiac tissue were collected during 2-3 hours after the echocardiographic evaluation as described previously in Protocol 1.

TABLE 1

| | Group | Treatment^{a} | Treatment Period |
|---|---|---|---|
| Protocol 1^{b} | 1 | Sham (n = 20) | Days 0-7 post- |
| | 2 | TAC + vehicle (n = 18) | TAC |
| | 3 | TAC + FBS (n = 16) | |
| Protocol 2^{c} | 4 | Sham + vehicle (n = 20) | Days 7-28 post- |
| | 5 | Sham + FBS (n = 12) | TAC |
| | 6 | Sham + allopurinol (n = 13) | |
| | 7 | TAC + vehicle (n = 20) | |
| | 8 | TAC + FBS (n = 21) | |
| | 9 | TAC + allopurinol (n = 17) | |

^{a}Sample (n) represents number of animals available to assess organ and body weights; subsets of equal or lesser numbers were used for echocardiographic and biochemical measurements.
^{b}Mice received either vehicle (250 μl vehicle) or febuxostat (FBS) (5 mg/kg/day) with the first dose given immediately after animals awoke from anesthesia post transverse aortic constriction (TAC) procedure; the last dose of FBS or vehicle was given ~1 hour before the echocardiogram.
^{c}FBS or allopurinol were given in drinking water at 0.05 mg/ml and 0.15 mg/ml, respectively, beginning 7 days after the TAC surgical procedure.

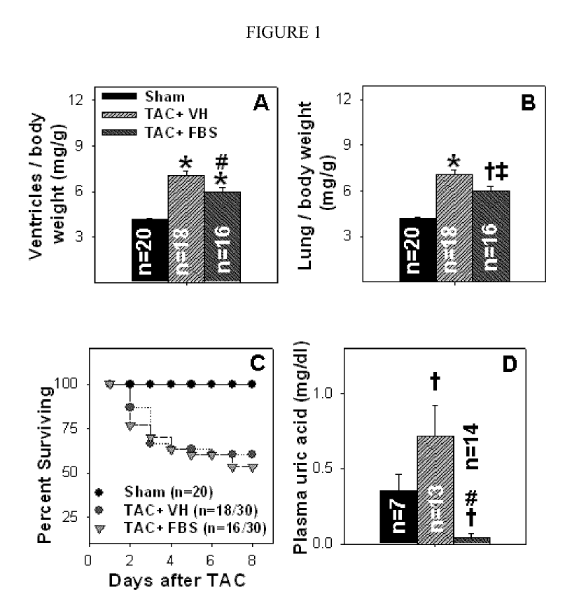

Minimally Invasive TAC Procedure. TAC was performed using the minimally invasive suprasternal approach described by Hu et al. (See, Hu et al., Am. J. Physiol. Heart Circ. Physiol., 3:H1261-9 (2003)). Mice were anesthetized with a mixture of 80 mg/kg ketamine and 30 mg/kg xylazine intraperitoneally. A horizontal incision was made at the level of the suprasternal notch to allow direct visualization of the transverse aorta without entering the pleural space. With the aid of a dissecting microscope, aortic constriction was performed by ligating the aorta between the right innominate artery and the left carotid artery over a 27 G) needle using 5-0 silk suture. The needle was immediately removed after ligation, leaving the aortic constriction in place. The incision was closed and the animals were allowed to recover. Analgesia was provided before the mice awoke from anesthesia. Sham surgery utilized the same procedure but without aortic ligation.
Echocardiography. Echocardiography was performed on mice anesthetized with 1.5% isoflurane by inhalation as previously by Zhang P., et al. and Lu, Z., et al. (See, Zhang, P., et al., Circ. Res., 7:1089-98 (2007) and Lu, Z., et al., Hypertension, 1:19-25 (2008)). LV end-diastolic diameter (LVEDD), LV end-systolic diameter (LVESD), and LV end-diastolic and end-systolic wall thicknesses (anterior and posterior) were measured using 2-dimensional guided M-mode echocardiography. LV fractional shortening (LVFS, %) was calculated as: LVFS=(LVEDD−LVESD)/LVEDD·100. LV ejection fraction (LVEF, %) was calculated by the cubic method: LVEF=[(LVEDD)³−(LVESD)³]/(LVEDD)³×100%.
Western Analysis. LV homogenates were clarified by centrifugation, and equal amounts of protein were loaded on 8-15% SDS-polyacrylamide gels and subjected to electrophoresis as described by Lu, Z., et al. (See, Lu, Z., et al., Hypertension, 1:19-25 (2008)). The separated protein bands were transferred to a HyBond nitrocellulose membrane, incubated with primary antibodies followed by horseradish peroxidase-labeled secondary antibody, and detected by enhanced chemiluminescent substrate (Amersham, Piscataway, N.J.). Light emission was detected by exposure to Fuji RX autoradiography film. Signal intensities were quantified using laser densitometry (Molecular Dynamics, Sunnyvale, Calif.). Primary antibodies against atrial natriuretic peptide (ANP) (Peninsula Laboratories Inc. San Carlos, Calif.), nitrotyrosine (Cayman Chemical, Ann Arbor, Mich.), pre-collagen I (Santa Cruz, Santa Cruz, Calif.), collagen III (Sigma, St. Louis, Mo.), total mammalian target of rapamycin (mTOR) (Cell Signaling, Danvers, Mass.), phosphorylated mTOR (p-mTOR^{Ser2488}) (Cell Signaling, Danvers, Mass.), Akt [protein kinase B (PKB)] (Santa Cruz, Santa Cruz, Calif.), phosphorylated Akt (p-Akt^{Ser473}) (Santa Cruz, Santa Cruz, Calif.), total extracellular signal-regulated kinase (Erk) (Cell Signaling, Danvers, Mass.), GAPDH (Santa Cruz, Santa Cruz, Calif.) and phosphorylated Erk (p-Erk^{Thr202/204}) (Cell Signaling, Danvers, Mass.) were used for Western blots.
Histological Staining and Measurement of Cardiac Myocyte Hypertrophy Tissue sections (8 μm thickness) of the central portion of the LV were stained with hematoxylin and eosin (H&E; Sigma, St. Louis, Mo.) for overall morphology or with Masson's trichrome (Sigma, St. Louis, Mo.) for myocardial fibrosis (See, Zhang, P., et al., Circ. Res., 7:1089-98 (2007)). FITC-conjugated wheat germ agglutinin (Invitrogen, Carlsbad, Calif.) was used to stain the cell membranes for determination of cardiac myocyte size. Cardiac myocyte size was assessed by measuring the short axis diameter of cardiomyocytes in cross section. For each heart, more than 100 cells in at least four representative cross sectional areas were measured, and data were averaged from four representative hearts in each treatment group.
Plasma Uric Acid Analysis. The concentration of uric acid in plasma was determined using Uric Acid Reagent (ThermoDMA, Louisville, Colo.) according to the manufacturer's protocol.
Data and Statistical Analyses. All values were expressed as mean±standard error of the mean (SEM). Statistical significance was defined as P<0.05. One-way analysis of variance (ANOVA) was used to test each variable for differences among the treatment groups with StatView (SAS Institute, Cary, N.C.). If the ANOVA demonstrated a significant effect, post hoc comparisons were made pairwise using Fisher's least significant difference test. When the data suggested a trend of difference between the groups, but did not show significance in the above tests, paired Student T test was also used as specially indicated. The Fisher exact test was used to compare mortality data among the treatment groups.
   Results—Protocol 1—Effects of Febuxostat Prevention Treatment for Eight Days (Beginning 30-60 Minutes after TAC)
Ventricular Hypertrophy, Pulmonary Congestion, Mortality and Uric acid. Body weight, ventricular weight, lung weight, the ratio of ventricular weight to body weight, and the ratio of lung weight to body weight are shown in FIG. 1 and Table 2.

TABLE 2

| Parameter | Sham | TAC + Vehicle | TAC + FBS |
|---|---|---|---|
| Body weight (g) | 23.5 ± 0.6 | 22.8 ± 0.7 | 23.4 ± 0.9 |
| Ventricle weight (mg) | 98 ± 3 | 159 ± 6* | 138 ± 7.0*^{,#} |
| Lung weight (mg) | 132 ± 4 | 195 ± 20* | 163 ± 16 |
| Heart rate (beats/min) | 478 ± 19 | 418 ± 14* | 415 ± 18* |
| LV fractional | 39.9 ± 1.6 | 22.1 ± 1.6* | 31.5 ± 2.2*^{,#} |
| shortening (%) | | | |
| Anterior wall thickness | 0.98 ± 0.03 | 1.14 ± 0.03* | 1.10 ± 0.04* |
| at end-systole (mm) | | | |
| Posterior wall thickness | 0.98 ± 0.03 | 1.13 ± 0.03* | 1.10 ± 0.03* |
| at end-systole (mm) | | | |
| Anterior wall thickness | 0.69 ± 0.02 | 0.90 ± 0.02* | 0.84 ± 0.02* |
| at end-diastole (mm) | | | |
| Posterior wall thickness | 0.69 ± 0.02 | 0.90 ± 0.02* | 0.83 ± 0.02*^{,#} |
| at end-diastole (mm) | | | |

*P < 0.05 as compared to sham group.
^{#}P < 0.05 as compared to TAC + vehicle group.

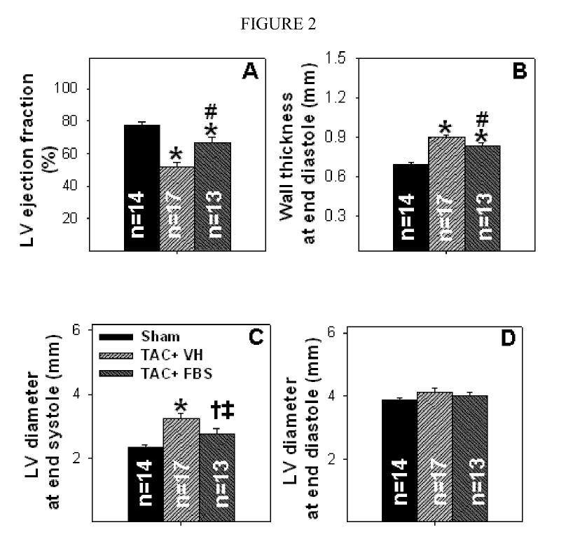

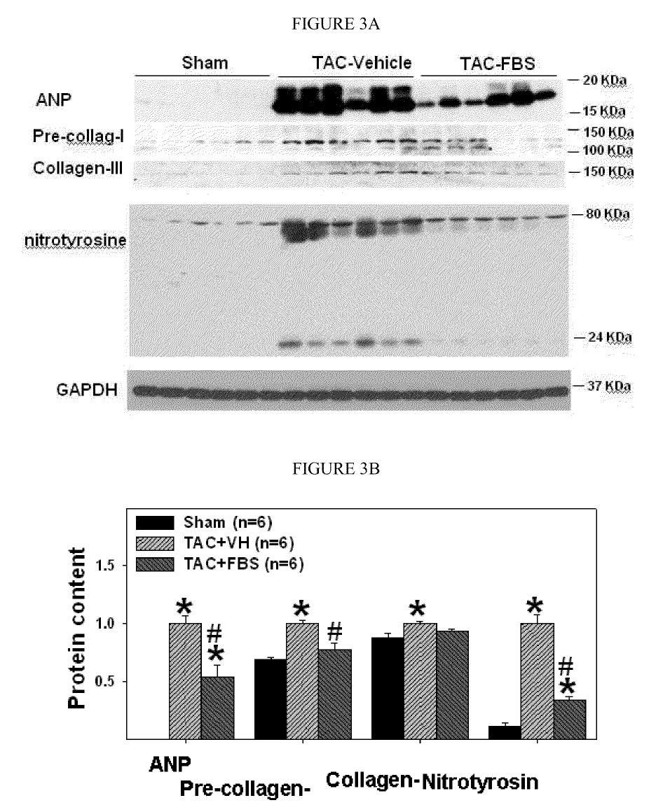

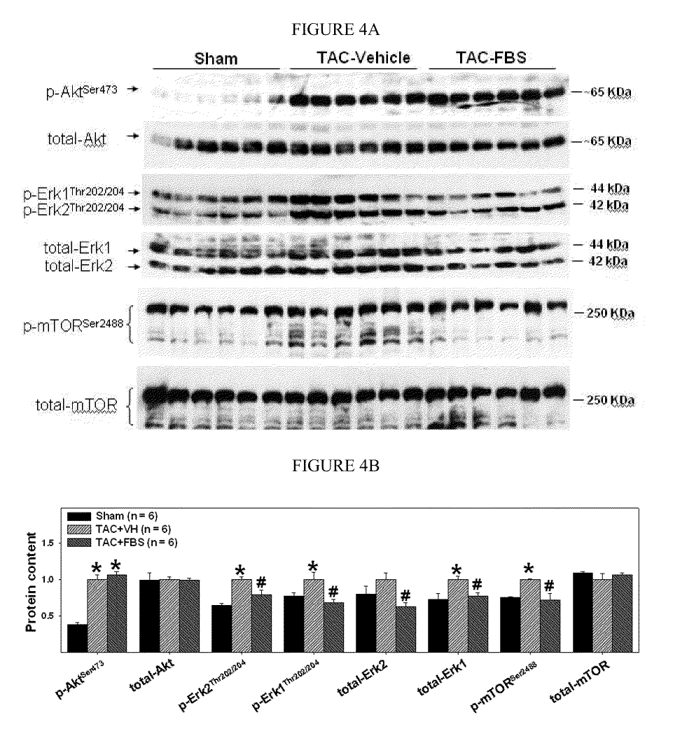

Eight days after TAC, the surviving animals had significant ventricular hypertrophy and pulmonary congestion. The degree of ventricular hypertrophy, assessed by the ratio of ventricular mass to body weight, was reduced by approximately 15% with febuxostat treatment (P<0.05) (See, FIG. 1A). Febuxostat treatment also decreased the degree of pulmonary congestion, assessed by the ratio of lung weight to body weight, by about 18%, although the difference was only statistically significant as compared to the TAC vehicle control group by using a paired Student t test (See, FIG. 1B). TAC resulted in significant mortality (mostly in the first four days after surgery; 12 out of 30 mice died in the TAC vehicle control group), and this was not affected by febuxostat treatment (14 out of 30 mice died) (See, FIG. 1C). TAC significantly increased plasma uric acid. Febuxostat significantly decreased plasma uric acid, and the uric acid level in the febuxostat-treated animals tended to be less than in the untreated sham animals (See, FIG. 1D), indicating that febuxostat treatment caused effective xanthine oxidase (XO) inhibition.
Left Ventricular Dilation and Dysfunction. In vehicle-treated animals, at 8 days after TAC, LV ejection fraction (See, FIG. 2A) and fractional shortening (See, Table 2) were significantly decreased, while LV wall thickness (See, Table 2) was increased. Febuxostat treatment significantly attenuated the TAC-induced increase of LV end-diastolic wall thickness (See, FIG. 2B) and also attenuated the decreases of LV fractional shortening and ejection fraction following TAC (See, FIG. 2A, Table 2). Moreover, febuxostat treatment showed a trend toward attenuating the TAC-induced increase of LV diameter at end-systole (it was statistically significant by the Student's t test) (See, FIG. 2C). Febuxostat had no significant effect on LV diameter at end-diastole (See, FIG. 2D). These findings indicate that early treatment with febuxostat protected against the LV hypertrophy and dysfunction that occurred in response to systolic overload.
Myocyte Hypertrophy and Ventricular Fibrosis. TAC significantly increased cardiac myocyte diameter from 13.2±0.2 μm in the sham group to 17.8±0.3 μm in the vehicle-treated TAC group, whereas febuxostat significantly attenuated the TAC-induced increase of cardiomyocyte diameter to 16.3±0.3 μm (p<0.05). This is consistent with the finding of less ventricular hypertrophy in the febuxostat-treated TAC group. TAC significantly increased myocardial fibrosis from 2.6±0.29% in the sham group to 12.2±3.0% in the vehicle-treated TAC group, whereas febuxostat tended to decrease the TAC-induced increase of myocardial fibrosis to 8.3±2.% (p=0.05).
Biochemical Analysis. Febuxostat treatment significantly attenuated the TAC-induced increase of myocardial ANP (See, FIG. 3), a biochemical marker of myocardial hypertrophy and heart failure (See, Gavin, A D., et al., Heart, 6:749-53 (2005)). In addition, febuxostat significantly attenuated the TAC-induced increases of myocardial pre-collagen I and collagen III content (See, FIG. 3), suggesting that febuxostat treatment limited ventricular collagen synthesis. Furthermore, febuxostat significantly blunted the TAC-induced increase of nitrotyrosine, a biochemical marker of myocardial oxidative stress (See, Giordano, F J., J. Clin. Invest., 3:500-8 (2005)).
Myocardial p-mTOR and p-Erk^{Thr202/204}. Systolic overload-induced myocardial hypertrophy is associated with activation of the PI3 kinase-Akt/PKB and mitogen-activated protein kinase signaling pathways, which play crucial roles in control of cell growth. Therefore, myocardial levels of total Akt, p-Akt^{Ser473}, total mTOR, p-mTOR^{S2488} (a downstream target of Akt), total Erk, and p-Erk^{Thr202/204} were determined by Western blots. TAC caused significant increases of p-Akt^{Ser473}, p-mTOR^{Ser2488}, t-Erk, and p-Erk^{Thr202/204}, but had no effect on t-Akt and t-mTOR (See, FIG. 4). Treatment with febuxostat significantly attenuated the TAC-induced increases of p-mTOR^{Ser2488}, t-Erk, and p-Erk^{Thr202/204} but not of t-Akt and p-Akt^{Ser473} (See, FIG. 4), indicating that febuxostat blunted the TAC-induced activation of the mTOR and Erk signaling pathways.

### Results—Protocol 2—Effects of Febuxostat or Allopurinol Treatment for Three Weeks (Beginning 7 Days After TAC)

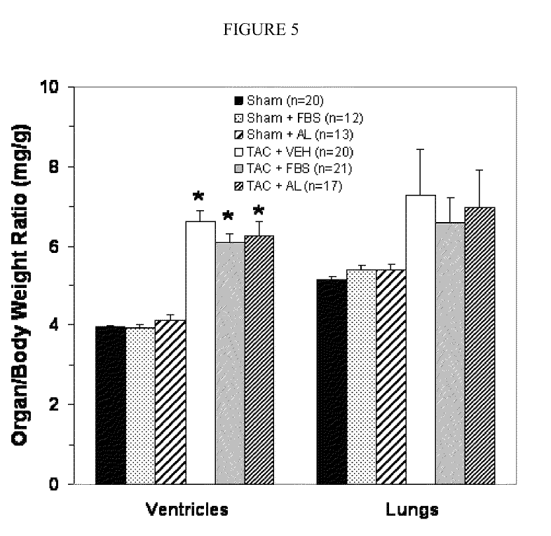

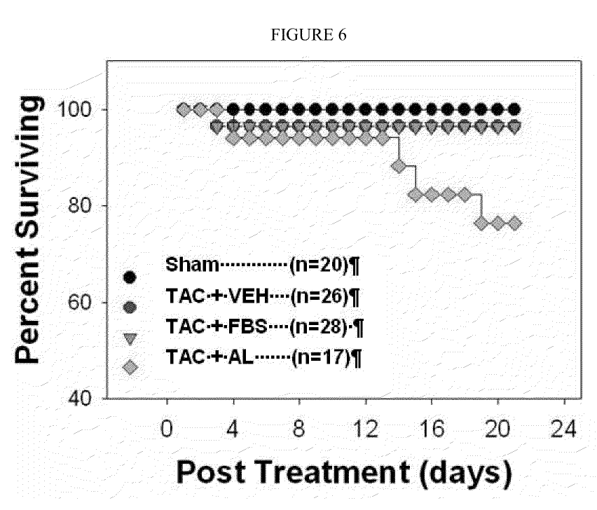

Ventricular Hypertrophy, Pulmonary Congestion, Mortality and Uric acid. Administration of FBS or AL had no significant effects on body weight, ventricular weight, lung weight, ratio of ventricular weight to body weight, and ratio of lung weight to body weight in the sham groups (FIG. 5, Table 3). There was no significant mortality over the three-week treatment period in TAC mice treated with vehicle (2 out 26 mice died, 92% survival rate) or FBS (1 out 28 mice died, 96% survival rate), but in TAC animals treated with allopurinol (AL), the survival rate dropped to 76% (4 out 17 died) (See, FIG. 6).

TABLE 3

| Parameter | Treatment | Sham | TAC |
|---|---|---|---|
| Body weight (g) | vehicle | 25.0 ± 0.4 | 25.2 ± 0.6 |
| | FBS | 24.9 ± 0.4 | 25.8 ± 0.4 |
| | allopurinol | 24.4 ± 0.6 | 24.8 ± 0.6 |
| Ventricle weight (mg) | vehicle | 99 ± 1 | 166 ± 6^{a} |
| | FBS | 98 ± 1 | 158 ± 6^{a} |
| | allopurinol | 100 ± 3 | 153 ± 6^{a} |
| Ventricle/body weight (mg/g) | vehicle | 3.96 ± 0.03 | 6.62 ± 0.29^{a} |
| | FBS | 3.94 ± 0.07 | 6.11 ± 0.21^{a} |
| | allopurinol | 4.13 ± 0.12 | 6.27 ± 0.36^{a} |
| Lung weight (mg) | vehicle | 129 ± 2 | 176 ± 20 |
| | FBS | 135 ± 2 | 170 ± 17 |
| | allopurinol | 131 ± 3 | 166 ± 16 |
| Lung/body weight (mg/g) | vehicle | 5.18 ± 0.06 | 7.28 ± 1.15 |
| | FBS | 5.42 ± 0.11 | 6.59 ± 0.64 |
| | allopurinol | 5.40 ± 0.14 | 6.98 ± 0.94 |
| Heart rate (beats/min) | vehicle | 501 ± 20 | 472 ± 13 |
| | FBS | 504 ± 19 | 454 ± 11 |
| | allopurinol | 490 ± 16 | 463 ± 10 |
| LV end-systolic diameter (mm) | vehicle | 2.25 ± 0.17 | 3.27 ± 0.11^{a} |
| | FBS | 1.94 ± 0.10 | 2.99 ± 0.10^{a} |
| | allopurinol | 2.22 ± 0.07 | 3.09 ± 0.10^{a} |
| LV end-diastolic diameter (mm) | vehicle | 3.85 ± 0.10 | 4.37 ± 0.09^{a} |
| | FBS | 3.74 ± 0.08 | 4.21 ± 0.08^{a} |
| | allopurinol | 3.71 ± 0.06 | 4.21 ± 0.08^{a} |
| LV ejection fraction (%) | vehicle | 78.4 ± 3.3 | 57.9 ± 1.9^{a} |
| | FBS | 85.5 ± 1.6^{a} | 63.9 ± 1.9^{a} |
| | allopurinol | 77.7 ± 2.1 | 60.2 ± 2.2^{a} |
| LV fractional shortening (%) | vehicle | 41.9 ± 3.7 | 25.3 ± 1.1^{a} |
| | FBS | 48.3 ± 2.2^{a} | 29.3 ± 1.3^{a} |
| | allopurinol | 40.1 ± 2.0 | 26.8 ± 1.4^{a} |
| Anterior wall thickness at end- | vehicle | 1.04 ± 0.05 | 1.17 ± 0.03 |
| systole (mm) | FBS | 1.18 ± 0.04 | 1.19 ± 0.02^{a} |
| | allopurinol | 1.11 ± 0.04 | 1.20 ± 0.03^{a} |
| Posterior wall thickness at end- | vehicle | 1.04 ± 0.05 | 1.16 ± 0.03 |
| systole (mm) | FBS | 1.18 ± 0.04^{a} | 1.18 ± 0.02^{a} |
| | allopurinol | 1.11 ± 0.03 | 1.20 ± 0.03^{a} |
| Anterior wall thickness at end- | vehicle | 0.69 ± 0.01 | 0.84 ± 0.03^{a} |
| diastole (mm) | FBS | 0.72 ± 0.02 | 0.85 ± 0.02^{a} |
| | allopurinol | 0.74 ± 0.01 | 0.87 ± 0.02^{a} |
| Posterior wall thickness at end- | vehicle | 0.70 ± 0.01 | 0.85 ± 0.02^{a} |
| diastole (mm) | FBS | 0.72 ± 0.02 | 0.84 ± 0.02^{a} |
| | allopurinol | 0.74 ± 0.01 | 0.88 ± 0.02^{a} |

^{a}significant difference relative to sham + vehicle group.

In the survived animals, chronic TAC resulted in a significant increase of ventricular weight and tended to increase lung weight (See, FIG. 5, Table 3). Administration of FBS or AL beginning seven days after TAC had no significant effect on the ratio of ventricular weight to body weight or on the ratio of lung weight to body weight (See, FIG. 5), suggesting that after the onset of cardiac hypertrophy and heart failure, febuxostat or allopurinol treatment had no significant effects on TAC-induced ventricular hypertrophy.

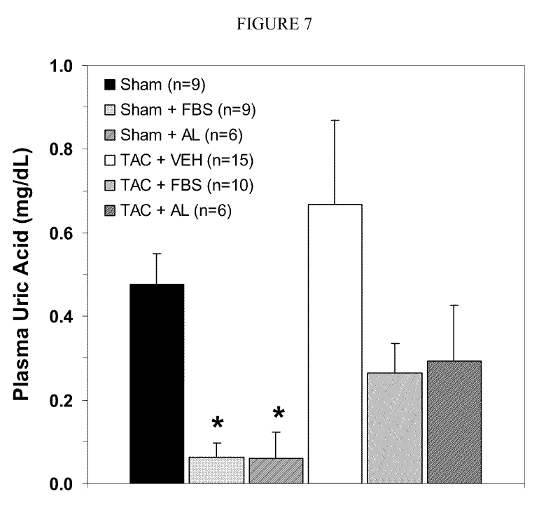

Both FBS and AL significantly decreased plasma uric acid in the three sham-operated groups (See, FIG. 7). They both also decreased plasma uric acid in TAC animals to a similar extent, but these changes did not reach statistical significance (See, FIG. 7).

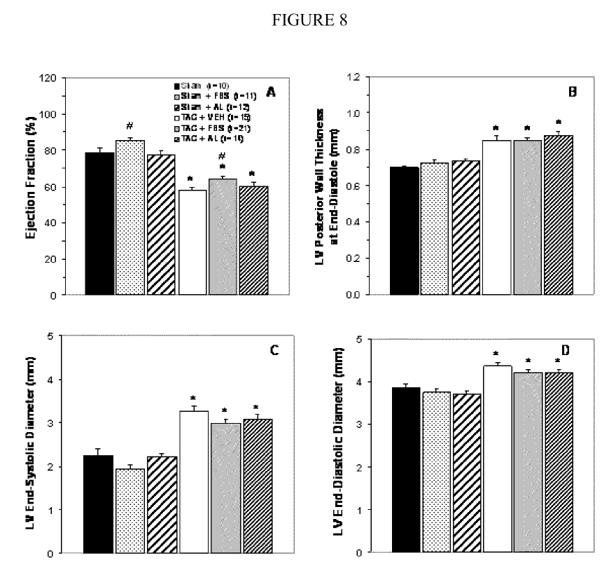

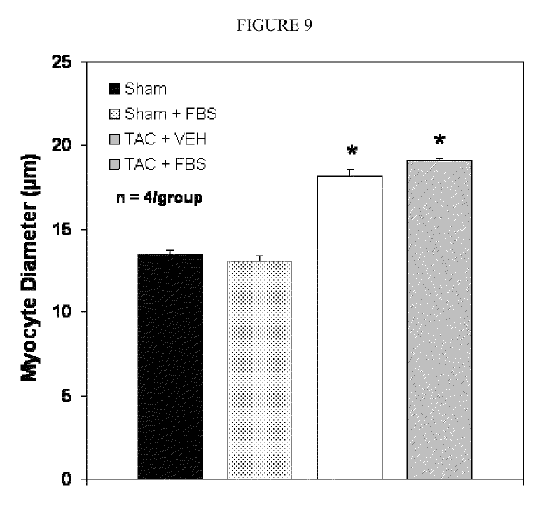

Average body weight increased 3.18±0.94, 2.93±0.42 and 3.38±0.37 g in mice treated with vehicle, FBS or allopurinol, respectively, during the three-week treatment period after TAC. These results indicate that FBS or AL did not affect the growth rate of the mice following TAC, suggesting that water and food intake were not affected.
Left Ventricular Dilation and Dysfunction The effects of FBS or AL on LV function and dimensions as measured by echocardiography are detailed in Table 3 and FIG. 8. In sham-operated animals, FBS resulted in a small increase in LV ejection fraction (˜9%, See, FIG. 8A) and fractional shortening (˜15%), but did not change LV end-diastolic diameter and end-systolic diameter or end-diastolic wall thickness (Table 2). In sham-operated animals, AL had no effect on any of the parameters evaluated. Although FBS had no effect on TAC-induced ventricular hypertrophy, it did induce a small improvement of LV ejection fraction (˜10% increase) and LV fractional shortening as compared with animals treated with vehicle (See, FIG. 8A). FBS tended to attenuate the TAC-induced increase of LV end-systolic diameter (P=NS), which correlates with the finding of improved shortening fraction (See, FIG. 8C). LV end-diastolic wall thickness and end-diastolic diameter were unchanged after FBS treatment (See, FIG. 8B and FIG. 8D). Similar to that observed in sham-operated animals, AL administration in TAC mice had no effect on LV function and dimensions as compared to vehicle-treated TAC mice (See, FIG. 8).
Myocyte Hypertrophy and Ventricular Fibrosis Histological staining demonstrated that TAC resulted in significant ventricular fibrosis, which was not affected by either FBS or AL. FBS also had no effect on TAC-induced cardiac myocyte hypertrophy (See, FIG. 9), which is consistent with the results on ventricular dimensions as measured by echocardiography. The effect of AL on cardiac myocytes was not measured since AL had no effects on both LV function and dimensions as measured by echocardiography.

### Discussion of Results

These studies demonstrated that administration of FBS shortly after chronic pressure overload (which mimics chronic hypertension) produced by TAC attenuated the pressure overload-induced cardiac hypertrophy, remodeling and LV dysfunction. This means that early treatment of febuxostat can reduce the progression of cardiac hypertrophy, cardiac remodeling or LV dysfunction induced by pressure overload. Febuxostat's beneficial effects on cardiac structure and function may be caused the reduction in the TAC-induced phosphorylation of mTOR and ERK, which plays crucial roles in control cell growth and contributes to the cardiac hypertrophy and remodeling process, and the increase of myocardial ANP, a biochemical marker of cardiac hypertrophy and heart failure. When FBS administration was delayed for seven days after TAC, the effects were significantly diminished as compared to treatment started soon after TAC. Treatment with FBS after the establishment of cardiac hypertrophy and dysfunction had very modest effects on TAC-induced ventricular dysfunction and no effect on hypertrophy.

Although administration of allopurinol beginning seven days after TAC resulted in a similar decrease of plasma uric acid, it did not produce any beneficial effects on TAC-induced LV dysfunction. Moreover, allopurinol tended to increase mortality as compared to control animals. Therefore, allopurinol had no beneficial, and perhaps even deleterious, effect as treatment of established cardiac hypertrophy and LV dysfunction in this animal model. The different effects of febuxostat and allopurinol may are due to differences in selectivity (e.g. febuxostat only inhibits xanthine oxidoreductase, while allopurinol can inhibit xanthine oxidoreductase and other enzymes in the purine and pyrimidine metabolism pathways) or chemical structure between these two compounds (e.g. allopurinol is a purine analog and febuxostat is a non-purine xanthine oxidoreductase inhibitor).

### Example 2

### Chronic Xanthine Oxidase Inhibition Following Myocardial Infarction in Rabbits

### Materials and Methods

Animals Male New Zealand white rabbits (1-2 kg; Clerco Research Farms, Cincinnati, Ohio, USA) were housed in separate cages in an environmentally controlled facility. The animals were subjected to circadian light-dark cycles, maintained on standard rabbit chow (2030C, Harlan Teklad, Madison, Wis., USA) and provided water ad libitum.

The procedures used in this study were reviewed and approved by the Institutional Animal Care and Use Committee (IACUC) of QTest Labs for compliance with regulations and current accepted practices. All applicable sections of the Final Rules of the Animal Welfare Act regulations (Section 9 CFR) and the Public Health Service Policy on Humane Care and Use of Laboratory Animals (OPRR, NIH, 1986) were followed. At the end of the study, all animals were humanely euthanized in accordance with accepted guidelines of the American Veterinary Medical Association (See, American Veterinary Medicine Association (AVMA), 2000 Report of the AVMA Panel on Euthanasia, The Journal of the American Veterinary Medical Association, 218(5):669-696 (2001)).
Drugs Febuxostat and allopurinol (Sigma-Aldrich, St. Louis, Mo., USA) were each formulated as a suspension in 0.5% methylcellulose (Sigma-Aldrich, St. Louis, Mo.) as the vehicle. The febuxostat oral dose of 10 mg/kg was estimated to provide a similar plasma level of febuxostat as the clinically efficacious dose (See, Khosravan et al., Clinical Pharmacokinetics, 45(8):821-841 (2006)). This dose was shown to be biologically active in rabbits as it caused a significant reduction in serum uric acid level (unpublished observations, 1998). Allopurinol was given orally at 60 mg/kg. A similar daily oral dose of allopurinol has been shown previously to be efficacious in a rabbit model of acute myocardial ischemia and reperfusion injury with significant myocardial ATP preservation, suggesting a sufficient inhibition of XO activity (See, Godin, D. V., et al., Biochemical Pharmacology, 36(13):2101-2107 (1987)).
Surgical Procedures Myocardial infarction (MI) was produced by permanent coronary artery ligation. The rabbits were anesthetized with an intramuscular injection of ketamine (35 mg/kg)/xylazine (5 mg/kg) and pre-surgery enrofloxacin (12 mg/kg). Isoflurane (1-2%) was given during the surgery as required to maintain a deep level of anesthesia and the animals were allowed to breathe 100% oxygen. A limb lead electrocardiogram (ECG) was obtained to monitor sinus rhythm, heart rate, and J-point deviation. The rabbit was placed in dorsal recumbency, and the sternum was exposed by blunt dissection and then incised with scissors. Following incision of the pericardial sac, a ligature was placed around the major branch of the left coronary artery, approximately superior to the midpoint between the starting point of the artery and the cardiac apex, using a 4-0 monofilament polypropylene suture with taper point needle. Myocardial ischemia was confirmed by regional cyanosis of the myocardial surface and ST-segment elevation on the ECG. The sternum was closed, leaving the pericardial sac as it was. The muscle layers, subcutaneous layer and skin were then closed. Ketofen (1 mg/kg) and buprenorphine (0.03 mg/kg/8 hour) were administered subcutaneously, and the rabbits were placed on a heating pad, covered with a towel, and observed continuously until awake. Post-operative medications given subcutaneously to the rabbits included enrofloxacin (12 mg/kg, b.i.d.), ketofen (0.03 mg/kg, b.i.d.), and buprenorphine (5 mg/kg, t.i.d. to q.i.d.) for 1 to 3 days as needed.
Experimental Procedures Rabbits were surgically altered (Day 0) to produce MI-induced heart failure and assigned to one of four treatment groups: the vehicle/control group (n=8) received vehicle for 49 consecutive days (7 weeks), the febuxostat/early treatment group (n=8) received febuxostat (10 mg/kg) for 49 consecutive days (7 weeks), the febuxostat/delayed treatment group (n=7) received vehicle for 21 consecutive days (3 weeks) and then febuxostat (10 mg/kg) starting on Day 22 (3 weeks post-surgery) for 28 consecutive days (4 weeks), and the allopurinol/delayed treatment group (n=8) received vehicle for 21 consecutive days and then allopurinol (60 mg/kg) starting on Day 22 (3 weeks post-surgery) for 28 consecutive days (4 weeks). All treatments were started on Day 1 (i.e. the day after surgery) and administered by oral gavage (2 mL/kg). The animals were fasted overnight prior to each day of dosing.

On Day 0 (baseline, pre-surgery), and on Days 21 (end of week 3) and 49 (end of week 7) at approximately one hour after dosing, the rabbits were sling restrained for ECG acquisition. The animals were then anesthetized using low doses of ketamine (35 mg/kg) and xylazine (5 mg/kg) administered intramuscularly, and a 2D-directed M mode echocardiogram (ECHO) (Cypress™; Acuson/Siemens, Malvern, Pa., USA) of the left ventricle was obtained to document ejection fraction (EF), shortening fraction (SF), and other dimensional parameters.

Following completion of acquisition of ECHO data on Day 49, rabbits were euthanized with sodium pentobarbital (240 mg, i.v.) and the hearts were removed from the chest. Post mortem pathology was performed and the hearts were weighed and preserved in 10% neutral buffered formalin.
Morphometric Analysis of Left Ventricular Fibrosis After fixation, the heart was trimmed into five blocks horizontally from the base to the apex with equal thickness and each block was embedded in paraffin. Five cross-sectional slides of the ventricles representing each of the five blocks were prepared for histology. The slides were stained with trichrome to facilitate visualization of collagen. Each slide was digitally photographed through a microscope and converted to a computer image with SPOT advanced software (Diagnostic Instruments, Sterling Heights, Mich., USA). Using Metamorph software (Molecular Devices Corp., Sunnyvale, Calif., USA), the following structures of each slide were manually traced with a cursor: the outer circumference of the left ventricle, the left ventricular cavity, and the circumference of the infarct. Metamorph automatically calculated the area within each trace. The sum of each area from all intact sections for each heart was determined, and relative percent infarct size for each heart was calculated using the following formula: 100×(sum of infarct area)/(sum of left ventricular area−sum of left ventricular cavity). No measurements were performed on cross sections which were not intact; the number of slides evaluated per heart ranged from 3 to 5 slides.
Data and Statistical Analyses The following ECHO parameters, reported as the average of three cardiac cycles, were obtained: left ventricular posterior wall dimension during diastole (LVPWd), left ventricular posterior wall dimension during systole (LVPWs), end diastolic volume (EDV), left ventricular internal dimension during diastole (LVIDd), left ventricular internal dimension during systole (LVIDs), shortening fraction [SF=100 (LVIDd−LVIDs)/LVIDd], and ejection fraction {EF=100 [(LVIDd)³−(LVIDs)³)]/(LVIDd)³}.

For each of the ECHO parameters (SF, EF, LVPWd, LVPWs, LVIDd, LVIDs, and EDV), summary statistics [mean and standard error of the mean (SEM)] were computed for the baseline values for each treatment group. The percent change from baseline (pre-surgery) values to post-surgery values measured on Days 21 and 49 were also computed for each parameter and treatment group. Pair wise comparisons between the vehicle/control group and each of the other treatment groups were made using contrast statements within the framework of an ANOVA model. Within each treatment group, a paired t-test was used to test whether the mean changes from baseline value to the values at Days 21 and 49 were different from zero.

The heart and body weights, heart/body weight ratios, infarct size and left ventricular wall thickness were also compared between the vehicle/control group and each of the three other treatment groups using an ANOVA model.

All statistical tests were two-tailed at the 0.05 level of significance. Comparisons yielding p values less than or equal to 0.05 were reported as statistically significant.

### Results

Left Ventricular Dilation and Dysfunction by Echocardiogram Baseline ECHO data for all treatment groups are presented in Table 4.

TABLE 4

| | Treatment Group | | | |
|---|---|---|---|---|
| | Vehicle/ | FBS/Early | FBS/Delayed | AL/Delayed |
| Parameter | Control | Treatment | Treatment | Treatment |
| SF (%) | 40 ± 1.5 | 34 ± 1.4* | 36 ± 1.2 | 40 ± 1.7 |
| EF (%) | 74 ± 1.8 | 68 ± 1.8* | 71 ± 1.7 | 74 ± 1.9 |
| LVPWd (cm) | 0.29 ± 0.01 | 0.26 ± 0.02 | 0.32 ± 0.02 | 0.31 ± 0.02 |
| LVPWs (cm) | 0.47 ± 0.03 | 0.49 ± 0.02 | 0.44 ± 0.02 | 0.46 ± 0.02 |
| LVIDd (cm) | 1.30 ± 0.04 | 1.25 ± 0.04 | 1.11 ± 0.04* | 1.29 ± 0.05 |
| LVIDs (cm) | 0.79 ± 0.04 | 0.82 ± 0.03 | 0.70 ± 0.02 | 0.77 ± 0.03 |
| EDV (mL) | 2.28 ± 0.22 | 1.94 ± 0.25 | 1.42 ± 0.13* | 2.21 ± 0.21 |

Values are presented as the mean ± SEM; n = 8;
*p < 0.05 compared to vehicle/control.
SF, shortening fraction;
EF, ejection fraction;
LVPWd, left ventricular posterior wall dimension during diastole;
LVPWs, left ventricular posterior wall dimension during systole;
LVIDd, left ventricular internal dimension during diastole,
LVIDs, left ventricular internal dimension during systole;
EDV, end diastolic volume.
Vehicle/Control: vehicle (0.5% methylcellulose, 2 mL/kg/day) was orally administered for 49 days starting 1 day after coronary occlusion.
FBS/Early Treatment: febuxostat (FBS, 10 mg/kg/day) was orally administered for 49 days starting 1 day after coronary occlusion.
FBS/Delayed Treatment: vehicle (2 mL/kg/day) was orally administered for 21 days starting 1 day after coronary occlusion followed by daily oral FBS (10 mg/kg/day) for 28 days.
AL/Delayed Treatment: vehicle (2 mL/kg/day) was orally administered for 21 days starting 1 day after coronary occlusion followed by daily oral allopurinol (AL, 60 mg/kg/day) for 28 days.

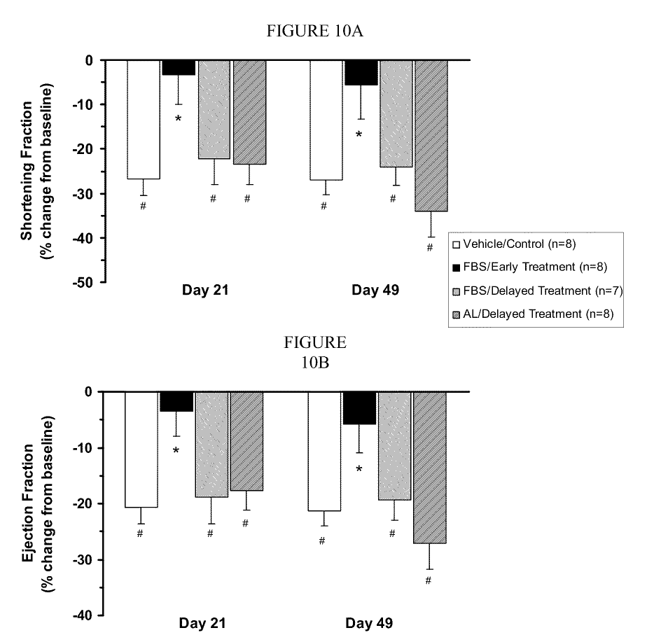

The effects of early or delayed treatment with febuxostat or allopurinol on ECHO parameters are summarized in FIGS. 10-13. In general, the baseline values were similar among the groups except that the left ventricular shortening and ejection fractions were slightly lower in the febuxostat early treatment group and the left ventricular internal dimension and volume at diastole were slightly lower in the febuxostat delayed treatment group. To allow for these minor differences in baseline, all values on Day 21 and 49 post-surgery were expressed as the percent change from baseline for all groups.

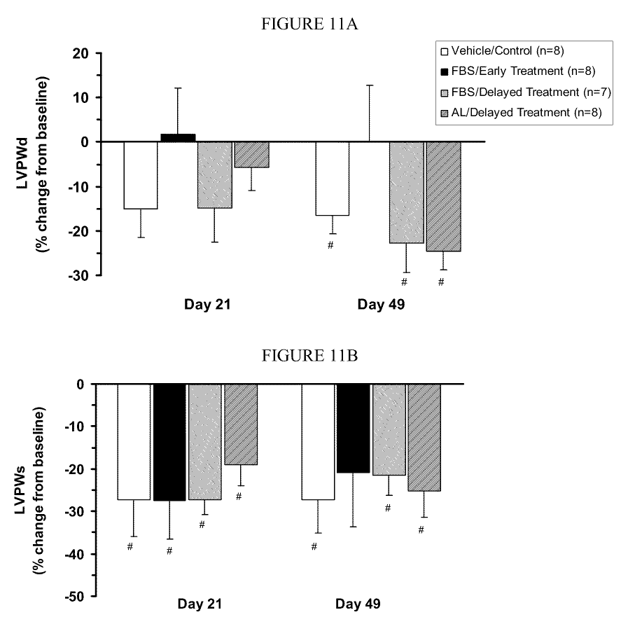

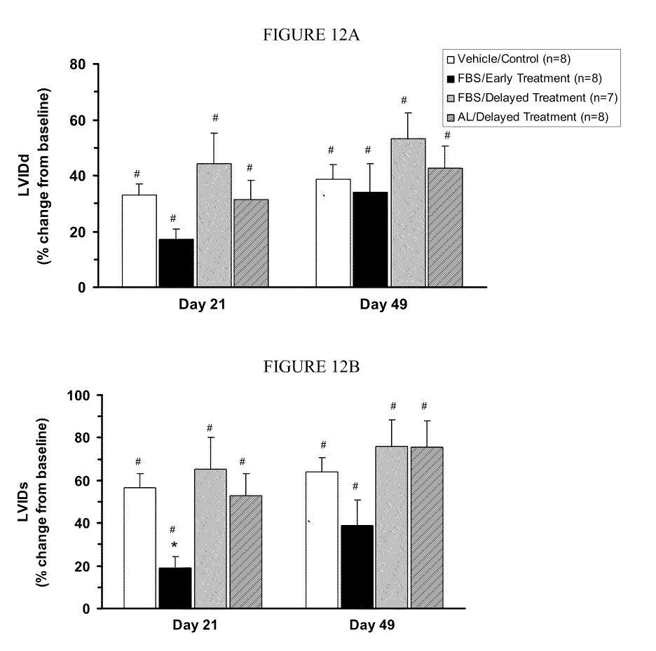

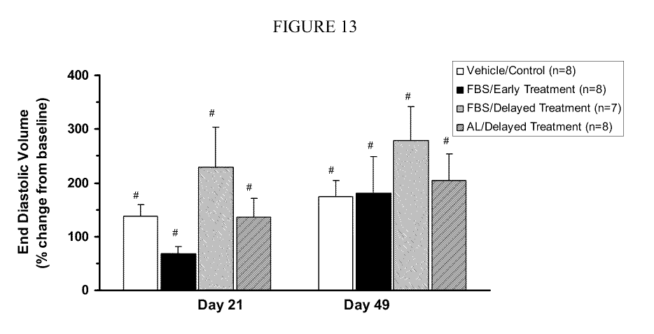

In the vehicle/control group, rabbits developed heart failure by Day 21 after coronary occlusion. This is demonstrated by a significant reduction in both shortening fraction (SF, −26.72±3.84%) and ejection fraction (EF, −20.62±3.06%) of the left ventricle as compared to the baseline (FIG. 10). In addition, there was significant thinning of the left ventricular posterior wall (during systole) (FIG. 11B) and increases in left ventricular internal dimensions (during both diastole and systole) (FIG. 12) and end diastolic volume (FIG. 13), all of which are all hallmarks of failing hearts. These characteristics of left ventricular dysfunction observed in this study are consistent with those reported previously by others using a similar rabbit model (See, Pye et al., Cardiovascular Research, 31(6):873-881 (1996), Pennock et al., American Journal of Physiology. Heart and Circulatory Physiology, 273(4):h2018-H2029 (1997)).

Early treatment with febuxostat starting one day after coronary artery ligation significantly lessened the reduction of shortening fraction (FIG. 10A) and ejection fraction (FIG. 10B) when compared to the vehicle/control group on Day 21, and these findings were still apparent on Day 49. In fact, in the febuxostat early treatment group, both of these values on Days 21 and 49 were not significantly different from the baseline. These results suggest that treatment with febuxostat shortly after the myocardial ischemic insult can offer cardiac functional protection. However, the shortening and ejection fractions (as percent change from baseline) for the febuxostat or allopurinol delayed treatment groups were not significantly different compared to the vehicle/control group on Days 21 and 49, indicating that treatment with either febuxostat or allopurinol after the establishment of heart failure will no longer offer cardiac functional benefit.

Correspondingly, there was no reduction in the left ventricular posterior wall dimension during diastole from baseline on both Days 21 and 49 in the febuxostat early treatment group (FIG. 11A). In addition, there was overall less of an increase in the left ventricular internal dimensions during diastole and systole (LVIDd and LVIDs, respectively) and end diastolic volume as compared to the vehicle/control group on Day 21, with the increase in LVIDs reaching statistical significance (FIGS. 12 and 13). Relative to the vehicle/control group, early and delayed treatment with an XO inhibitor had no effect on LV posterior wall dimention during systole.
Heart Weight and Heart/Body Weight Ratio The effects of febuxostat or allopurinol on the heart and body weights and heart/body weight ratio on Day 49 are summarized in Table 5.

TABLE 5

| | Treatment Group | | | |
|---|---|---|---|---|
| Weight on | Vehicle/ | FBS/Early | FBS/Delayed | AL/Delayed |
| Day 49 | Control | Treatment | Treatment | Treatment |
| Heart Weight | 6.83 ± 0.40 | 6.38 ± 0.19 | 6.65 ± 0.27 | 6.59 ± 0.30 |
| (g) | | | | |
| Body Weight | 2.63 ± 0.06 | 2.55 ± 0.06 | 2.37 ± 0.13 | 2.70 ± 0.04 |
| (kg) | | | | |
| Heart/Body | 0.26 ± 0.01 | 0.25 ± 0.01 | 0.28 ± 0.01 | 0.24 ± 0.01 |
| Weight | | | | |
| Ratio (%) | | | | |

Values presented as the mean ± SEM; n = 8, except n = 7 in the FBS/Delayed Treatment group.
Vehicle/Control: vehicle (0.5% methylcellulose, 2 mL/kg/day) was orally administered for 49 days starting 1 day after coronary occlusion.
FBS/Early Treatment: febuxostat (FBS, 10 mg/kg/day) was orally administered for 49 days starting 1 day after coronary occlusion.
FBS/Delayed Treatment: vehicle (2 mL/kg/day) was orally administered for 21 days starting 1 day after coronary occlusion followed by daily oral FBS (10 mg/kg/day) for 28 days.
AL/Delayed Treatment: vehicle (2 mL/kg/day) was orally administered for 21 days starting 1 day after coronary occlusion followed by daily oral allopurinol (AL, 60 mg/kg/day) for 28 days.

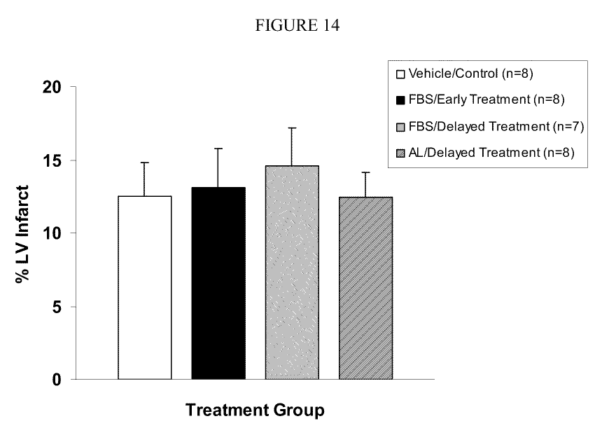

No statistically significant differences were found in these parameters between the drug-treated groups and the vehicle/control group. However, there was a small trend toward a lower heart weight in the febuxostat early treatment group relative to the vehicle/control group, which is consistent with the improvement in cardiac dimensions and function as determined by ECHO. Infarct Size There were no significant differences in infarct size between the four treatment groups (FIG. 14), indicating that none of the treatments had an impact on the amount of the infracted tissue in this permanent coronary artery-occlusion rabbit model.

### Discussion of Results

Left ventricular ejection fraction and shortening fraction are both considered “gold standards” of cardiac status, and a decrease in these parameters over time is predictive of mortality in heart failure patients (See, Lewis et al., J. Amer. College of Cardiol., 42(8):1446-1453 (2003)). The results of this study clearly show that early treatment with febuxostat, starting one day after coronary artery ligation in rabbits, preserved left ventricular function at 3 and 7 weeks post-ligation, with no significant reductions in ejection fraction or shortening fraction on both Days 21 and 49 relative to baseline, and the changes from baseline were significantly smaller than those in the vehicle-treated controls. In addition, these cardiac functional benefits were accompanied by moderately less severe changes in the left ventricular dimensional parameters than seen in the vehicle/control group. In contrast, when treatment with febuxostat or allopurinol was started after the establishment of heart failure (Day 21), no effects on cardiac functional or dimensional parameters were observed.

There is substantial evidence that oxidative stress participates in the pathophysiology of cardiac remodeling, hypertrophy and LV dysfunction; all of these, if left untreated, can likely lead to the development of heart failure (HF) (See, Giordano, F. J., J. Clin. Invest., 115(3):500-508 (2005) and Weir et al., The American Journal of Cardiology, 97 (10, Suppl. 1), 13-25 (2006)). XO, which generates reactive oxygen species as a product of purine metabolism, has been considered as an important contributor to oxidative stress in failing hearts (See, Giordano, F. J., J. Clin. Invest., 115(3):500-508 (2005)). Upregulation of the enzyme expression by various pro-inflammatory stimuli and hypoxia is central to the role of XO in HF. Ischemic insult to the myocardial tissue has also been documented to increase the activity of XO (See, Ungvari et al., Current Vas. Pharma., 3(3):221-229 (2005)).

The protective effects associated with early, chronic febuxostat treatment in this rabbit model provide additional support for the role of XO in the development of CHF post-MI. However, once CHF was established, XO inhibition no longer offered any significant protective effects. These results suggest that in rabbits, and perhaps in humans, inhibition of XO activity and its associated oxidative stress may only be of therapeutic benefit during the initial phase of left ventricular remodeling and cardiac functional deterioration after MI. Other proposed mediators of CHF development such as NADH oxidase, neutrophil infiltration, pro-inflammatory cytokines and neurohormonal factors (See, Giordano, F. J., J. Clin. Invest., 115(3):500-508 (2005)) may eventually overwhelm the contribution of XO, making it a less significant player during the later stages of CHF progression.

Febuxostat's non-purine structure is purported to afford greater tolerance in patients with renal dysfunction as it undergoes hepatic metabolism, in contrast to allopurinol which is mainly cleared by the kidneys (See, Schumacher, Jr., H. R., Expert Opin. On Invest. Drugs, 14(7):893-903 (2005)). The greater XO selectivity of febuxostat also avoids the cross-inhibition that allopurinol has on other purine and pyrimidine metabolism enzymes such as purine nucleoside phosphorylase and orotidine-5′-monophosphate decarboxylase (See, Takano et al., Life Sciences, 76(16):1835-1847 (2005)). The current study did not find any significant differences between the effects of chronic administration of allopurinol or febuxostat in rabbits with established heart failure. However, differences were observed in a pressure overload-induced cardiac hypertrophy and heart failure model in mice, where febuxostat had moderate beneficial effect on LV function and allopurinol appeared to be toxic when these compounds were administered after the onset of heart failure.

In conclusion, the results of this study suggest that chronic preventive treatment with febuxostat initiated shortly after MI could offer significant cardiac protection and reduce the progression of cardiac hypertrophy, remodeling and LV dysfunction, and thereby delay the onset of heart failure.

While the invention has been described by reference to certain presently preferred embodiments, it will be understood that modifications and variations thereof apparent to those skilled in the art are intended to be included within the scope of the invention.

What is claimed is:

1. A method for reducing the progression of at least one of cardiac hypertrophy, cardiac remodeling or left ventricular dysfunction following a cardiac insult in a subject in need of treatment thereof, the method comprising the step of:
administering to the subject in need of treatment thereof a therapeutically effective amount of at least one compound to reduce the progression of at least one of cardiac hypertrophy, cardiac remodeling or left ventricular dysfunction following a cardiac insult, wherein said at least one compound is a xanthine oxidoreductase inhibitor or a pharmaceutically acceptable salt thereof.

2. The method of claim 1, wherein the xanthine oxidoreductase inhibitor is selected from the group consisting of: 2-[3-cyano-4-(2-methylpropoxy)phenyl]-4-methylthiazole-5-carboxylic acid, 2-[3-cyano-4-(3-hydroxy-2-methylpropoxy)phenyl]-4-methyl-5-thiazolecarboxylic acid, 2-[3-cyano-4-(2-hydroxy-2-methylpropoxy)phenyl]-4-methyl-5-thiazolecarboxylic acid, 2-(3-cyano-4-hydroxyphenyl)-4-methyl-5-thiazolecarboxylic acid, 2-[4-(2-carboxypropoxy)-3-cyanophenyl]-4-methyl-5-thiazolecarboxylic acid, 1-(3-cyano-4-(2,2-dimethylpropoxy)phenyl)-1H-pyrazole-4-carboxylic acid, 1-3-cyano-4-(2,2-dimethylpropoxy)phenyl]-1H-pyrazole-4-carboxylic acid, pyrazolo [1,5-a]-1,3,5-triazin-4-(1H)-one, 8-[3-methoxy-4-(phenylsulfinyl)phenyl]-sodium salt (±), 3-(2-methyl-4-pyridyl)-5-cyano-4-isobutoxyphenyl)-1,2,4-triazole and a pharmaceutically acceptable salt thereof.

3. The method of claim 1, wherein the cardiac insult is myocardial infarction.

4. The method of claim 3, wherein the compound is administered to the subject within about 1 minute to about 16 days after the myocardial infarction.

5. The method of claim 1, wherein the cardiac insult is chronic hypertension.

6. A method for reducing the progression of at least one of cardiac hypertrophy, cardiac remodeling or left ventricular dysfunction following a cardiac insult in a subject in need of treatment thereof, the method comprising the step of:
administering to the subject in need of treatment thereof a therapeutically effective amount of at least one compound to delay the progression of at least one of cardiac hypertrophy, cardiac remodeling or left ventricular dysfunction following a cardiac insult, wherein said at least one compound is comprises the formula: 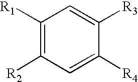
wherein R₁ and R₂ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, a phenylsulfinyl group or a cyano (—CN) group;
wherein R₃ and R₄ are each independently a hydrogen or A, B, C or D as shown below: 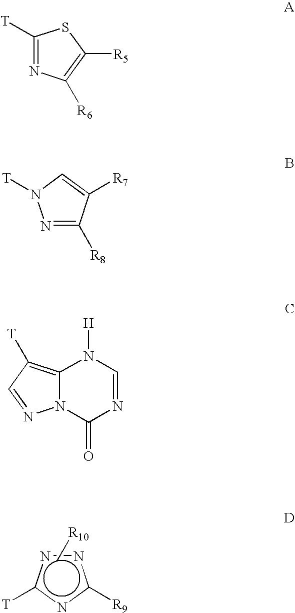
wherein T connects A, B, C or D to the aromatic ring shown above at R₁, R₂, R₃ or R₄,
wherein R₅ and R₆ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;
wherein R₇ and R₈ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;
wherein R₉ is an unsubstituted pyridyl group or a substituted pyridyl group; and
wherein R₁₀ is a hydrogen or a lower alkyl group, a lower alkyl group substituted with a pivaloyloxy group and in each case, R₁₀ bonds to one of the nitrogen atoms in the 1,2,4-triazole ring shown above.

7. The method of claim 6, wherein the compound is 2-[3-cyano-4-(2-methylpropoxy)phenyl]-4-methylthiazole-5-carboxylic acid or a pharmaceutically acceptable salt thereof.

8. The method of claim 6, wherein the compound is 2-[3-cyano-4-(3-hydroxy-2-methylpropoxy)phenyl]-4-methyl-5-thiazolecarboxylic acid or a pharmaceutically acceptable salt thereof.

9. The method of claim 6, wherein the compound is 2-[3-cyano-4-(2-hydroxy-2-methylpropoxy)phenyl]-4-methyl-5-thiazolecarboxylic acid or a pharmaceutically acceptable salt thereof.

10. The method of claim 6, wherein the compound is 2-(3-cyano-4-hydroxyphenyl)-4-methyl-5-thiazolecarboxylic acid or a pharmaceutically acceptable salt thereof.

11. The method of claim 6, wherein the compound is 2-[4-(2-carboxypropoxy)-3-cyanophenyl]-4-methyl-5-thiazolecarboxylic acid or a pharmaceutically acceptable salt thereof.

12. The method of claim 6, wherein the compound is 1-3-cyano-4-(2,2-dimethylpropoxy)phenyl]-1H-pyrazole-4-carboxylic acid or a pharmaceutically acceptable salt thereof.

13. The method of claim 6, wherein the compound is pyrazolo [1,5-a]-1,3,5-triazin-4-(1H)-one, 8-[3-methoxy-4-(phenylsulfinyl)phenyl]- sodium salt (±).

14. The method of claim 6, wherein the compound is 3-(2-methyl-4-pyridyl)-5-cyano-4-isobutoxyphenyl)-1,2,4-triazole or a pharmaceutically acceptable salt thereof.

15. The method of claim 6, wherein the cardiac insult is myocardial infarction.

16. The method of claim 15, wherein the compound is administered to the subject within about 1 minute to about 16 days after the myocardial infarction.

17. The method of claim 6, wherein the cardiac insult is chronic hypertension.

18. A method for reducing the progression of at least one of cardiac hypertrophy, cardiac remodeling or left ventricular dysfunction following a cardiac insult in a subject in need of treatment thereof, the method comprising the step of:
administering to the subject in need of treatment thereof a therapeutically effective amount of at least one compound to delay the progression of at least one of cardiac hypertrophy, cardiac remodeling or left ventricular dysfunction following a cardiac insult, wherein said at least one compound is comprises the formula: 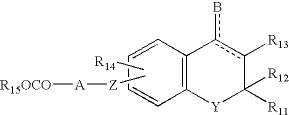
wherein R₁₁ and R₁₂ are each independently a hydrogen, a substituted or unsubstituted lower alkyl group, a substituted or unsubstituted phenyl, or R₁₁ and R₁₂ may together form a four- to eight-membered carbon ring together with the carbon atom to which they are attached;
wherein R₁₃ is a hydrogen or a substituted or unsubstituted lower alkyl group;
wherein R₁₄ is one or two radicals selected from a group consisting of a hydrogen, a halogen, a nitro group, a substituted or unsubstituted lower alkyl, a substituted or unsubstituted phenyl, —OR₁₆ and —SO₂NR₁₇R_{17′}, wherein R₁₆ is a hydrogen, a substituted or unsubstituted lower alkyl, a phenyl-substituted lower alkyl, a carboxymethyl or ester thereof, a hydroxyethyl or ether thereof, or an allyl; R₁₇ and R_{17′} are each independently a hydrogen or a substituted or unsubstituted lower alkyl;
wherein R₁₅ is a hydrogen or a pharmaceutically active ester-forming group;
wherein A is a straight or branched hydrocarbon radical having one to five carbon atoms;
wherein B is a halogen, an oxygen, or an ethylenedithio;
wherein Y is an oxygen, a sulfur, a nitrogen or a substituted nitrogen;
wherein Z is an oxygen, a nitrogen or a substituted nitrogen; and
the dotted line refers to either a single bond, a double bond, or two single bonds.

19. The method of claim 18, wherein the cardiac insult is myocardial infarction.

20. The method of claim 19, wherein the compound is administered to the subject within about 1 minute to about 16 days after the myocardial infarction.

21. The method of claim 18, wherein the cardiac insult is chronic hypertension.

22. A method for delaying the onset of heart failure symptoms or reducing the incidence of cardiovascular events following a cardiac insult in a subject in need of treatment thereof, the method comprising the step of:
administering to the subject in need of treatment thereof a therapeutically effective amount of at least one compound to delay the onset of heart failure symptoms or reducing the incidence of cardiovascular events, wherein said at least one compound is a xanthine oxidoreductase inhibitor or a pharmaceutically acceptable salt thereof.

23. The method of claim 22, wherein the xanthine oxidoreductase inhibitor is selected from the group consisting of: 2-[3-cyano-4-(2-methylpropoxy)phenyl]-4-methylthiazole-5-carboxylic acid, 2-[3-cyano-4-(3-hydroxy-2-methylpropoxy)phenyl]-4-methyl-5-thiazolecarboxylic acid, 2-[3-cyano-4-(2-hydroxy-2-methylpropoxy)phenyl]-4-methyl-5-thiazolecarboxylic acid, 2-(3-cyano-4-hydroxyphenyl)-4-methyl-5-thiazolecarboxylic acid, 2-[4-(2-carboxypropoxy)-3-cyanophenyl]-4-methyl-5-thiazolecarboxylic acid, 1-(3-cyano-4-(2,2-dimethylpropoxy)phenyl)-1H-pyrazole-4-carboxylic acid, 1-3-cyano-4-(2,2-dimethylpropoxy)phenyl]-1H-pyrazole-4-carboxylic acid, pyrazolo [1,5-a]-1,3,5-triazin-4-(1H)-one, 8-[3-methoxy-4-(phenylsulfinyl)phenyl]-sodium salt (±), 3-(2-methyl-4-pyridyl)-5-cyano-4-isobutoxyphenyl)-1,2,4-triazole and a pharmaceutically acceptable salt thereof.

24. The method of claim 22, wherein the cardiac insult is a myocardial infarction.

25. The method of claim 24, wherein the compound is administered to the subject within about 1 minute to about 16 days after the myocardial infarction.

26. The method of claim 22, wherein the cardiac insult is chronic hypertension.

27. A method for delaying the onset of heart failure symptoms or reducing the incidence of cardiovascular events following a cardiac insult in a subject in need of treatment thereof, the method comprising the step of:
administering to the subject in need of treatment thereof a therapeutically effective amount of at least one compound to delay the onset of heart failure symptoms or reducing the incidence of cardiovascular events, wherein said compound comprises the formula: 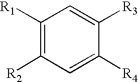
wherein R₁ and R₂ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, a phenylsulfinyl group or a cyano (—CN) group;
wherein R₃ and R₄ are each independently a hydrogen or A, B, C or D as shown below: 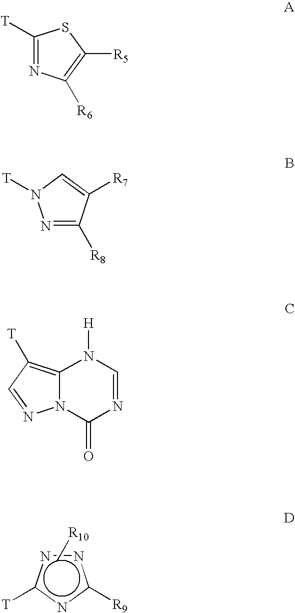
wherein T connects A, B, C or D to the aromatic ring shown above at R₁, R₂, R₃ or R₄,
wherein R₅ and R₆ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;
wherein R₇ and R₈ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;
wherein R₉ is an unsubstituted pyridyl group or a substituted pyridyl group; and
wherein R₁₀ is a hydrogen or a lower alkyl group, a lower alkyl group substituted with a pivaloyloxy group and in each case, R₁₀ bonds to one of the nitrogen atoms in the 1,2,4-triazole ring shown above.

28. The method of claim 27, wherein the compound is 2-[3-cyano-4-(2-methylpropoxy)phenyl]-4-methylthiazole-5-carboxylic acid or a pharmaceutically acceptable salt thereof.

29. The method of claim 27, wherein the compound is 2-[3-cyano-4-(3-hydroxy-2-methylpropoxy)phenyl]-4-methyl-5-thiazolecarboxylic acid or a pharmaceutically acceptable salt thereof.

30. The method of claim 27, wherein the compound is 2-[3-cyano-4-(2-hydroxy-2-methylpropoxy)phenyl]-4-methyl-5-thiazolecarboxylic acid or a pharmaceutically acceptable salt thereof.

31. The method of claim 27, wherein the compound is 2-(3-cyano-4-hydroxyphenyl)-4-methyl-5-thiazolecarboxylic acid or a pharmaceutically acceptable salt thereof.

32. The method of claim 27, wherein the compound is 244-(2-carboxypropoxy)-3-cyanophenyl]-4-methyl-5-thiazolecarboxylic acid or a pharmaceutically acceptable salt thereof.

33. The method of claim 27, wherein the compound is 1-3-cyano-4-(2,2-dimethylpropoxy)phenyl]-1H-pyrazole-4-carboxylic acid or a pharmaceutically acceptable salt thereof.

34. The method of claim 27, wherein the compound is pyrazolo [1,5-a]-1,3,5-triazin-4-(1H)-one, 8-[3-methoxy-4-(phenylsulfinyl)phenyl]- sodium salt (±).

35. The method of claim 27, wherein the compound is 3-(2-methyl-4-pyridyl)-5-cyano-4-isobutoxyphenyl)-1,2,4-triazole or a pharmaceutically acceptable salt thereof.

36. The method of claim 27, wherein the cardiac insult is myocardial infarction.

37. The method of claim 36, wherein the compound is administered to the subject within about 1 minute to about 16 days after the myocardial infarction.

38. The method of claim 27, wherein the cardiac insult is chronic hypertension.

39. A method for delaying the onset of heart failure symptoms or reducing the incidence of cardiovascular events following a cardiac insult in a subject in need of treatment thereof, the method comprising the step of:
administering to the subject in need of treatment thereof a therapeutically effective amount of at least one compound to delay the onset of heart failure symptoms or reducing the incidence of cardiovascular events, wherein said compound comprises the formula: 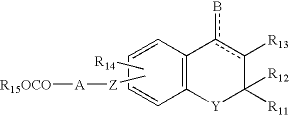
wherein R₁₁ and R₁₂ are each independently a hydrogen, a substituted or unsubstituted lower alkyl group, a substituted or unsubstituted phenyl, or R₁₁ and R₁₂ may together form a four- to eight-membered carbon ring together with the carbon atom to which they are attached;
wherein R₁₃ is a hydrogen or a substituted or unsubstituted lower alkyl group;
wherein R₁₄ is one or two radicals selected from a group consisting of a hydrogen, a halogen, a nitro group, a substituted or unsubstituted lower alkyl, a substituted or unsubstituted phenyl, —OR₁₆ and —SO₂NR₁₇R_{17′}, wherein R₁₆ is a hydrogen, a substituted or unsubstituted lower alkyl, a phenyl-substituted lower alkyl, a carboxymethyl or ester thereof, a hydroxyethyl or ether thereof, or an allyl; R₁₇ and R_{17′} are each independently a hydrogen or a substituted or unsubstituted lower alkyl;
wherein R₁₅ is a hydrogen or a pharmaceutically active ester-forming group;
wherein A is a straight or branched hydrocarbon radical having one to five carbon atoms;
wherein B is a halogen, an oxygen, or an ethylenedithio;
wherein Y is an oxygen, a sulfur, a nitrogen or a substituted nitrogen;
wherein Z is an oxygen, a nitrogen or a substituted nitrogen; and
the dotted line refers to either a single bond, a double bond, or two single bonds.

40. The method of claim 39, wherein the cardiac insult is a myocardial infarction.

41. The method of claim 40, wherein the compound is administered to the subject within about 1 minute to about 16 days after the myocardial infarction.

42. The method of claim 39, wherein the cardiac insult is chronic hypertension.
